(19) United States
(12) Patent Application Publication
(10) Pub. No.: US 2024/0181227 A1
(43) Pub. Date: Jun. 6, 2024

(54) SYSTEM FOR PREVENTING CATHETER BALLOON HYPERINFLATION

(71) Applicant: EDWARDS LIFESCIENCES CORPORATION, Irvine, CA (US)

(72) Inventor: Julio Agustin Isla Garcia, Irvine, CA (US)

(21) Appl. No.: 18/436,971

(22) Filed: Feb. 8, 2024

Related U.S. Application Data
(63) Continuation of application No. PCT/US2022/038906, filed on Jul. 29, 2022.
(60) Provisional application No. 63/231,482, filed on Aug. 10, 2021.
(60) Provisional application No. 63/231,500, filed on Aug. 10, 2021.

(51) Int. Cl. A61M 25/10; A61M 1/36

(52) U.S. Cl. CPC A61M 25/10186; A61M 1/3659; A61M 1/3666; A61M 25/10182; A61M 2205/3344

(57) ABSTRACT

A system includes an injection device that is configured to be fluidly coupled to the catheter and a sensing device that is configured to sense an injected volume of fluid in the inflatable balloon of the catheter. A controller is coupled by a wired or wireless communication link to the sensing device to receive an injected volume signal from the sensing device. The controller is configured to determine an actuation pressure based on the injected volume of the fluid in the inflatable balloon. A pressure relief valve is fluidly coupled to the catheter and coupled by a wired or wireless communication link to the controller. The controller is configured to adjust the actuation pressure of the pressure relief valve based on the injected volume of the fluid in the inflatable balloon.

### CROSS-REFERENCE TO RELATED APPLICATION(S)

This application n claims priority to International Application No. PCT/US2022/038906, filed on Jul. 29, 2022, and entitled “System for Preventing Catheter Balloon Hyperinflation,” which claims priority to U.S. Provisional Application No. 63/231,482, filed on Aug. 10, 2021, and entitled “System for Preventing Catheter Balloon Hyperinflation,” and to U.S. Provisional Application No. 63/231,500, filed on Aug. 10, 2021, and entitled “System for Preventing Catheter Balloon Hyperinflation,” the disclosures of which are incorporated by reference in their entireties.

### BACKGROUND

The present disclosure relates to pulmonary artery catheters, and in particular, to a system for preventing hyperinflation of inflatable balloons.

Pulmonary artery catheters, also known as Swan-Ganz catheters, can be advanced into the pulmonary artery of a patient to continuously monitor hemodynamic variables. Pulmonary artery catheters allow for the continuous sensing of flow, pressure, and oxygenation delivery and consumption. Specifically, pulmonary artery catheters can be used to determine the following hemodynamic variables: cardiac output (the volume of blood being pumped by the heart per unit of time), mixed venous oxygen saturation (measure of the relationship between oxygen delivery and oxygen consumption), stroke volume (the volume of blood ejected from the ventricle in each beat), systemic vascular resistance (the resistance that must be overcome to push blood through the circulatory system), right ventricular ejection fraction (the percentage of blood ejected from the ventricle with each beat), right ventricular end diastolic volume (the volume of blood in the ventricle at the end of the diastole), right atrial pressure (the blood pressure in the right atrium of the heart), pulmonary artery pressure (the blood pressure in the pulmonary artery), pulmonary artery occlusion pressure (an estimate of the blood pressure in the left atrium) (also known as the pulmonary wedge pressure), and diastolic pulmonary artery pressure (the blood pressure in the pulmonary artery at the end of the diastole).

The hemodynamic variables that are determined with pulmonary artery catheters can help in the diagnosis, monitoring, and treatment of the following: acute heart failure, severe hypovolemia, complex circulatory situations, medical emergencies, acute respiratory distress syndrome, gram negative sepsis, drug intoxication, acute renal failure, hemorrhagic pancreatitis, intra and post-operative management of high risk patients, history of pulmonary or cardiac disease, fluid shifts, management of high-risk obstetrical patients, diagnosed cardiac disease, toxemia, premature separation of placenta, cardiac output determinations, differential diagnosis of mitral regurgitation and ventricular septal rupture, and diagnosis of cardiac tamponade. Pulmonary artery catheters can also be used to monitor hemodynamic variables during the following procedures: coronary artery bypass graft, aortic valve replacement/repair, mitral valve replacement/repair, aortic valve conduit, aortic arch replacement, cardiogenic shock, acute mitral regurgitation, ventricular septal rupture, and pulmonary artery hypertension.

One of the risks associated with advancing a catheter into the pulmonary artery is pulmonary artery aneurysm or rupture. The complications from pulmonary artery aneurysm or rupture can happen anywhere between several minutes to months after the procedure. The treatment to mitigate the damage is complex and costly, involving angiography, CT, catheter embolization, and/or surgical resection. The damage can occur due to hyperinflation of the catheter balloon, repeated inflation, and flushing of the catheter while the balloon is inflated and wedged in the pulmonary artery.

### SUMMARY

A system for preventing hyperinflation of an inflatable balloon of a catheter includes an injection device that is configured to be fluidly coupled to the catheter to inject a fluid into the inflatable balloon of the catheter, and a sensing device that is configured to sense an injected volume of the fluid in the inflatable balloon of the catheter. A controller is coupled by a wired or wireless communication link to the sensing device to receive an injected volume signal from the sensing device that indicates the injected volume of the fluid in the inflatable balloon of the catheter. The controller is configured to determine an actuation pressure based on the injected volume of the fluid in the inflatable balloon. A pressure relief valve is fluidly coupled to the catheter and coupled by a wired or wireless communication link to the controller. The controller is configured to adjust the actuation pressure of the pressure relief valve based on the injected volume of the fluid in the inflatable balloon.

A method of preventing hyperinflation of an inflatable balloon of a catheter includes injecting a fluid into the inflatable balloon of the catheter with an injection device and measuring an injected volume of the fluid in the inflatable balloon of the catheter with a sensing device. An injected volume signal is sent from the sensing device to a controller, wherein the injected volume signal indicates the injected volume of the fluid in the inflatable balloon. The controller determines an actuation pressure based on the injected volume of the fluid in the inflatable balloon. The actuation pressure of a pressure relief valve is adjusted based on the injected volume of the fluid in the inflatable balloon.

### BRIEF DESCRIPTION OF THE DRAWINGS

FIG. 1A is a schematic view of a pulmonary artery catheter in a heart.

FIG. 1B is a top plan view of the pulmonary artery catheter.

FIG. 2 is a block diagram of a hyperinflation prevention system for preventing hyperinflation of an inflatable balloon of a pulmonary artery catheter.

FIG. 3A is a graph showing a pressure versus injected volume curve and a balloon diameter versus injected volume curve for an inflatable balloon that is unloaded.

FIG. 3B is a graph showing a pressure versus injected volume curve and a balloon diameter versus injected volume curve for the inflatable balloon in a soft, tight cylinder.

FIG. 3C is a graph showing a pressure versus injected volume curve and a balloon diameter versus injected volume curve for the inflatable balloon in a soft, loose cylinder.

FIG. 3D is a graph showing a pressure versus injected volume curve and a balloon diameter versus injected volume curve for the inflatable balloon in a stiff, loose cylinder.

FIG. 4 is a graph showing a threshold curve and the pressure versus injected volume curve for an inflatable balloon in a stiff, loose vessel.

FIG. 5A is a graph showing a pressure versus injected volume curve and a balloon diameter versus injected volume curve for an inflatable balloon in a soft, loose vessel.

FIG. 5B is a graph showing a pressure versus injected volume curve and a balloon diameter versus injected volume curve for an inflatable balloon in a stiff, loose vessel.

FIG. 6 is a graph showing a threshold curve and a pressure versus injected volume curve for an inflatable balloon that is being pulled while inflated.

FIG. 7 is a partial cross-sectional view of a first embodiment of a valve mechanism.

FIG. 8A is a partial cross-sectional view of the first embodiment of the valve mechanism in a closed position.

FIG. 8B is a partial cross-sectional view of the first embodiments of the valve mechanism in an open position.

FIG. 9 is a partial cross-sectional view of a second embodiment of a valve mechanism.

FIG. 10A is a partial cross-sectional view of the second embodiment of the valve mechanism in a closed position.

FIG. 10B is a partial cross-sectional view of the second embodiment of the valve mechanism in an open position.

FIG. 11 is a side view of a delivery mechanism.

FIG. 12A is a side view of a first embodiment of a strain gauge sensor.

FIG. 12B is a cross-sectional view of the first embodiment of the strain gauge sensor, taken along line 12B-12B of FIG. 12A.

FIG. 13A is a side view of a second embodiment of a strain gauge sensor.

FIG. 13B is a cross-sectional view of the second embodiment of the strain gauge sensor, taken along line 13B-13B of FIG. 13A.

FIG. 14 is a cross-sectional view of a first embodiment of an inflatable balloon.

FIG. 15 is a cross-sectional view of a second embodiment of an inflatable balloon.

FIG. 16 is a cross-sectional view of a third embodiment of an inflatable balloon.

FIG. 17 is a cross-sectional view of a fourth embodiment of an inflatable balloon.

FIG. 18A is a side view of a fifth embodiment of an inflatable balloon.

FIG. 18B is a cross-sectional view of the fifth embodiment of an inflatable balloon, taken along line 18B-18B of FIG. 18A.

FIG. 19A is a schematic view showing a heterogenous inflatable balloon on a catheter as it is being inflated.

FIG. 19B is a schematic view showing the heterogenous inflatable balloon on the catheter as a center of the heterogenous inflatable balloon reaches its maximum stretchability.

FIG. 19C is a schematic view showing the heterogenous inflatable balloon on the catheter as a proximal end and a distal end of the heterogenous inflatable balloon expand.

FIG. 20 is a schematic view showing a heterogenous inflatable balloon anchored to a catheter.

FIG. 21 is a schematic top view of a first embodiment of a thread pattern for a heterogenous inflatable balloon.

FIG. 22 is a schematic cross-sectional view of a second embodiment of a thread pattern for a heterogenous inflatable balloon.

### DETAILED DESCRIPTION

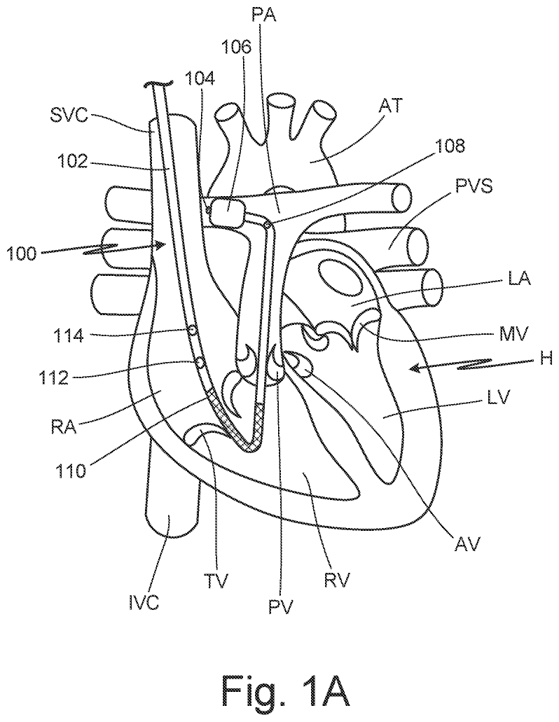

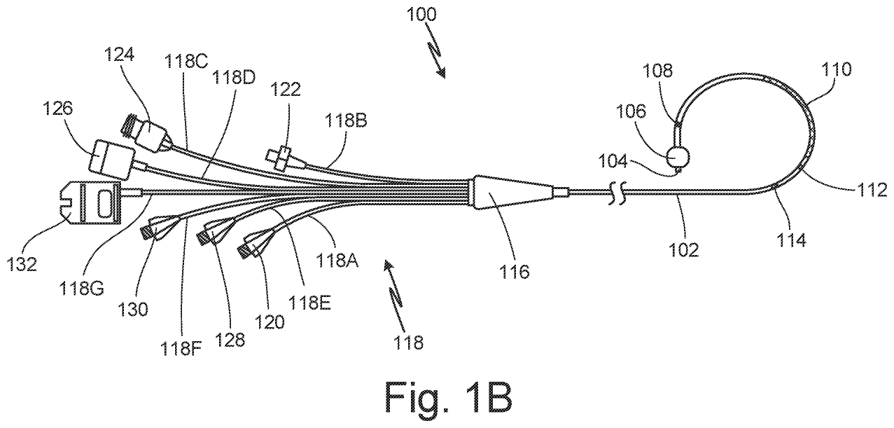

FIG. 1A is a schematic view of pulmonary artery catheter 100 in heart H. FIG. 1B is a top plan view of pulmonary artery catheter 100. Pulmonary artery catheter 100 includes catheter body 102, distal port 104, inflatable balloon 106, thermistor 108, thermal filament 110, proximal injectate port 112, volume infusion port 114, catheter body junction 116 (shown in FIG. 1B), extension tubes 118 (including extension tubes 118A-118G) (shown in FIG. 1B), distal port hub 120 (shown in FIG. 1B), balloon inflation valve 122 (shown in FIG. 1B), thermistor connector 124 (shown in FIG. 1B), thermal filament connector 126 (shown in FIG. 1B), proximal injectate lumen hub 128 (shown in FIG. 1B), volume infusion port hub 130 (shown in FIG. 1B), and optical module connector 132 (shown in FIG. 1B). FIG. 1A also shows heart H, right atrium RA, right ventricle RV, left atrium LA, left ventricle LV, tricuspid valve TV, pulmonary valve PV, mitral valve MV, aortic valve AV, superior vena cava SVC, inferior vena cava IVC, pulmonary artery PA, pulmonary veins PVS, and aorta AT.

Pulmonary artery catheter 100 (also called a Swan-Ganz catheter) can be advanced to a patient's pulmonary artery PA for continuous monitoring of flow, pressure, and oxygen delivery and consumption. When paired with a cardiac output monitor, pulmonary artery catheter 100 can provide a comprehensive hemodynamic profile of a patient. A patient's hemodynamic status can be tracked using the comprehensive hemodynamic profile and continuous data to assist physicians in early evaluation of the patient's cardiac status. Specifically, pulmonary artery catheter 100 can be used to determine the following parameters when paired with a cardiac monitor: cardiac output (CO), mixed venous oxygen situation (SvO₂), stroke volume (SV), systemic vascular resistance (SVR), right ventricular ejection fraction (RVEF), right ventricular end diastolic volume (RVEDV), right atrial pressure (RAP), pulmonary artery pressure (PAP), pulmonary artery occlusion pressure (PAOP), and diastolic pulmonary artery pressure (PADP).

Pulmonary artery catheter 100 includes catheter body 102. Distal port 104 is positioned at a distal end of catheter body 102. Distal port 104 can be used to monitor the pulmonary artery pressure and allows mixed venous blood samples to be taken from pulmonary artery PA for the assessment of oxygen transport balance and the calculation of oxygen consumption, oxygen utilization coefficient, and intrapulmonary shunt fraction. Inflatable balloon 106 is positioned adjacent to distal port 104 near the distal end of catheter body 102. Inflatable balloon 106 can be inflated when pulmonary artery catheter 100 is positioned in heart H and floated into pulmonary artery PA to sense a patient's hemodynamic variables.

Pulmonary artery catheter 100 further includes thermistor 108 positioned proximal of inflatable balloon 106. Thermistor 108 senses a temperature of pulmonary artery PA when inflatable balloon 106 is positioned in pulmonary artery PA. The temperature readings are used to calculate cardiac output measurements. Thermal filament 110 is positioned proximal of thermistor 108. When inflatable balloon 106 is positioned in pulmonary artery PA, thermal filament 110 is positioned in right atrium RA and right ventricle RV of heart H. Proximal injectate port 112 is positioned proximal of thermal filament 110. When inflatable balloon 106 is positioned in pulmonary artery PA, proximal injectate port 112 is positioned in right atrium RA of heart H. Proximal injectate port 112 can be used to determine a right atrial or central venous pressure, to take blood samples, to infuse medicine to right atrium RA, or to inject a fluid bolus into heart H for cardiac output measurement. Volume infusion port 114 is positioned proximal of proximal injectate port 112. When inflatable balloon 106 is positioned in pulmonary artery PA of heart H, volume infusion port 114 is positioned in right atrium RA of heart H. Volume infusion port 114 provides direct access to right atrium RA and allows for continuous infusion into right atrium RA of heart H. In alternate embodiments, pulmonary artery catheter 100 can include electrodes for right atrial, right ventricular, or right A-V sequential temporary transveous pacing.

Catheter body junction 116 is positioned at a proximal end of catheter body 102. Extending from catheter body junction 116 are plurality of extension tubes 118. There are seven extension tubes 118 shown in FIG. 1B, but any number of extension tubes 118 can extend from catheter body junction 116 in alternate embodiments. Each of extension tubes 118 extends from catheter body junction 116 to a connector at a proximal end of each extension tube 118.

Distal port hub 120 is positioned at a proximal end of extension tube 118A. Distal port hub 120 is fluidly connected to distal port 104 through a lumen extending through catheter body 102 and through extension tube 118A. Balloon inflation valve 122 is positioned at a proximal end of extension tube 118B. Balloon inflation valve 122 is fluidly connected to inflatable balloon 106 through a lumen extending through catheter body 102 and through extension tube 118B. An injection device, such as a syringe, can be connected to balloon inflation valve 122 to inject a fluid into inflatable balloon 106.

Thermistor connector 124 is positioned at a proximal end of extension tube 118C. Thermal filament connector 126 is positioned at a proximal end of extension tube 118D. Proximal injectate lumen hub 128 is positioned at a proximal end of extension tube 118E. Proximal injectate lumen hub 128 is fluidly connected to proximal injectate port 112 through a lumen extending through catheter body 102 and through extension tube 118E. Volume infusion port hub 130 is positioned at a proximal end of extension tube 118F. Volume infusion port hub 130 is fluidly connected to volume infusion port 114 through a lumen extending through catheter body 102 and through extension tube 118F. Optical module connector 132 is positioned at a proximal end of extension tube 118G.

Pulmonary artery catheter 100 can be inserted into a large vein in the patient, typically the internal jugular veins, the subclavian veins, the femoral veins, or the antecubital fossa veins. Pulmonary artery catheter 100 is then advanced through the vascular systems and into the right atrium RA of heart H. The passage of pulmonary artery catheter 100 to the right atrium RA can be monitored with dynamic pressure readings from distal port 104 on pulmonary artery catheter 100 and/or with fluoroscopy. Once the distal end of pulmonary artery catheter 100 is positioned in right atrium RA of heart H, inflatable balloon 106 is inflated. Inflatable balloon 106 will then float from right atrium RA, through tricuspid valve TV, into and through right ventricle RV, through pulmonary valve PV, and into pulmonary artery PA. Inflatable balloon 106 will float through pulmonary artery PA until it wedges in pulmonary artery PA, as shown in FIG. 1A.

Pulmonary artery catheter 100 can determine hemodynamic parameters as inflatable balloon 106 is floated through heart H. Once inflatable balloon 106 is wedged in pulmonary artery PA, pulmonary artery catheter 100 can determine further hemodynamic parameters. Once the hemodynamic parameters are determined, inflatable balloon 106 can be deflated and pulmonary artery catheter 100 can be pulled from the patient.

One of the risks associated with advancing pulmonary artery catheter 100 into pulmonary artery PA is pulmonary artery aneurysm or rupture. The complications from pulmonary artery aneurysm or rupture can happen due to hyperinflation of inflatable balloon 106 of catheter 100 or a physician pulling catheter 100 when inflatable balloon 106 is still inflated. Hyperinflation prevention system 200 is disclosed herewith to prevention hyperinflation of inflatable balloon 106.

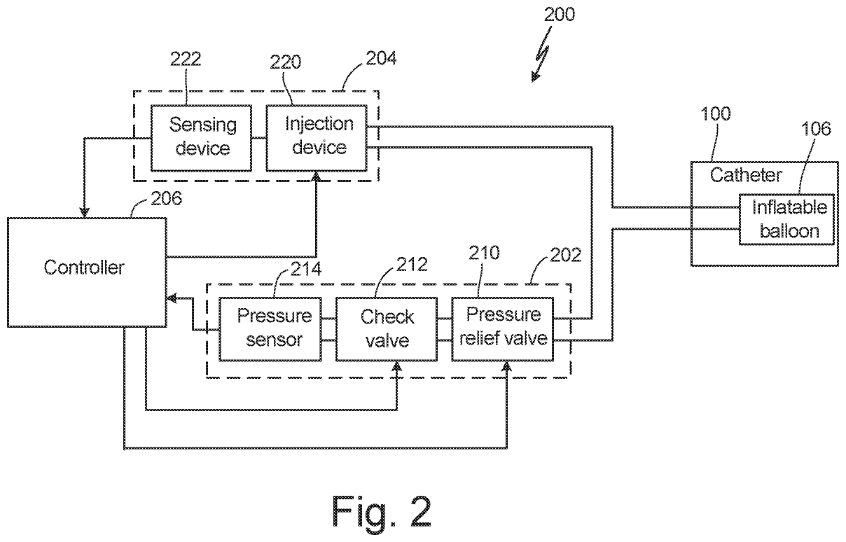

FIG. 2 is a block diagram of hyperinflation prevention system 200 for preventing hyperinflation of inflatable balloon 106 of pulmonary artery catheter 100. FIG. 2 shows catheter 100 and hyperinflation prevention system 200. Catheter 100 includes inflatable balloon 106. Hyperinflation prevention system 200 includes valve mechanism 202, delivery mechanism 204, and controller 206. Valve mechanism 202 includes pressure relief valve 210, check valve 212, and pressure sensor 214. Delivery mechanism 204 includes injection device 220 and sensing device 222.

Catheter 100 and inflatable balloon 106 are schematically shown in FIG. 2, but have the structure and design as shown in and discussed in reference to FIGS. 1A-1B. Hyperinflation prevention system 200 will be discussed here as preventing hyperinflation of inflatable balloon 106 of catheter 100, but hyperinflation prevention system 200 can be used with any suitable catheter to prevent hyperinflation of any inflatable balloon in alternate embodiments.

Hyperinflation prevention system 200 is configured to prevent hyperinflation of inflatable balloon 106 of catheter 100. Hyperinflation prevention system 200 includes valve mechanism 202, delivery mechanism 204, and controller 206. Valve mechanism 202 and delivery mechanism 204 are both fluidly coupled to inflatable balloon 106 of catheter 100. Valve mechanism 202 and delivery mechanism 204 are both coupled by a wired or wireless communication link to controller 206.

Valve mechanism 202 includes pressure relief valve 210 that is configured to prevent hyperinflation of inflatable balloon 106. Valve mechanism 202 also includes check valve 212 that is configured to prevent negative pressure in inflatable balloon 106. Valve mechanism 202 further includes pressure sensor 214 that is configured to sense a pressure signal that represents a pressure in inflatable balloon 106. The pressure signal sensed by pressure sensor 214 can be the pressure in inflatable balloon 106 or any other parameter that can be used by pressure sensor 214 or controller 206 to determine the pressure in inflatable balloon 106. In the embodiment shown in FIG. 2, pressure relief valve 210, check valve 212, and pressure sensor 214 integrally form valve mechanism 202. In alternate embodiments, pressure relief valve 210, check valve 212, and pressure sensor 214 can be separate components that are fluidly coupled. Further, pressure relief valve 210 and check valve 212 can be an integral unit, pressure relief valve 210 and pressure sensor 214 can be an integral unit, and check valve 212 and pressure sensor 214 can be an integral unit in alternate embodiments.

Delivery mechanism 204 includes injection device 220 that is configured to inject a fluid into inflatable balloon 106 of catheter 100. In the embodiment shown in FIG. 2, injection device 220 is configured to inject air into inflatable balloon 106 of catheter 100. In alternate embodiments, any suitable fluid can be injected into inflatable balloon 106 of catheter 100. Delivery mechanism 204 also includes sensing device 222 that is configured to sense an injected volume signal that represents an injected volume of fluid that has been injected into inflatable balloon 106 of catheter 100. Sensing device 222 can be fluidly, electrically, or mechanically coupled to delivery mechanism 204. The injected volume signal sensed by sensing device 222 can be the injected volume of fluid in inflatable balloon 106 or any other parameter that can be used by sensing device 222 or controller 206 to determine the injected volume of fluid in inflatable balloon 106. For example, sensing device 222 can be a force sensor that senses a force in sensing device 222 that can be converted to an injected volume of fluid in inflatable balloon 106 by sensing device 222 or controller 206. In the embodiment shown in FIG. 2, injection device 220 and sensing device 222 integrally form delivery mechanism 204. In alternate embodiments, injection device 220 and sensing device 222 can be separate components that are fluidly, electrically, or mechanically coupled.

Valve mechanism 202 and delivery mechanism 204 are electrically and/or communicatively coupled to controller 206 by a wired or wireless communication link. Controller 206 can include one or more processors and computer-readable memory encoded with instructions that, when executed by the one more processors, cause controller 206 to operate in accordance with techniques described herein. For example, controller 206 can include one or more processors and computer-readable memory encoded with instructions that, when executed by the one or more processors, cause controller 206 to operate in accordance with techniques described herein. Examples of the one or more processors include any one or more of a microprocessor, a controller, a digital signal processor (DSP), an application specific integrated circuit (ASIC), a field-programmable gate array (FPGA), or other equivalent discrete or integrated logic circuitry. Computer-readable memory of controller 206 can be configured to store information within controller 206 during operation. The computer-readable memory can be described, in some examples, as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). Computer-readable memory of controller 206 can include volatile and non-volatile memories. Examples of volatile memories can include random access memories (RAM), dynamic random access memories (DRAM), static random access memories (SRAM), and other forms of volatile memories. Examples of non-volatile memories can include magnetic hard discs, optical discs, flash memories, or forms of electrically programmable memories (EPROM) or electrically erasable and programmable (EEPROM) memories.

Controller 206 is coupled by a wired or wireless communication link to pressure relief valve 210, check valve 212, and pressure sensor 214 of valve mechanism 202. Controller 206 is configured to receive the pressure signal from pressure sensor 214. Controller 206 is configured to send a signal to pressure relief valve 210 to dynamically adjust pressure relief valve 210. In the embodiment shown in FIG. 2, controller 206 is configured to send a signal to check valve 212 to actuate check valve 212. In alternate embodiments, check valve 212 is not coupled to controller 206. Further, controller 206 is coupled by a wired or wireless communication link to injection device 220 and sensing device 222 of delivery mechanism 204. Controller 206 is configured to receive the injected volume signal from sensing device 222. In the embodiment shown in FIG. 2, controller 206 is configured to send a signal to injection device 220 to adjust injection device 220 to withdraw fluid from catheter 100. In alternate embodiments, injection device 220 is not coupled to controller 206.

Check valve 212 of valve mechanism 202 is configured to prevent negative pressure in inflatable balloon 106 to avoid damage to inflatable balloon 106 and to reset injection device 220. Inflatable balloon 106 may experience negative pressure when pressure relief valve 210 is actuated and/or when injection device 220 is actuated. In the embodiment shown in FIG. 2, check valve 212 is electrically actuated. When a negative pressure exists in inflatable balloon 106, controller 206 will send a signal to check valve 212 to actuate check valve 212. When check valve 212 is actuated, it will open catheter 100 to an ambient surrounding catheter 100 to prevent negative pressure in catheter 100. In alternate embodiments, check valve 212 can be automatically mechanically actuated when a negative pressure exists in inflatable balloon 106.

Pressure relief valve 210 is configured to prevent hyperinflation of inflatable balloon 106 to avoid damaging the patient's vessels. Pressure relief valve 210 is electrically adjustable. Controller 206 can send a signal to pressure relief valve 210 to dynamically adjust the actuation pressure needed to actuate pressure relief valve 210. When pressure relief valve 210 is actuated, it will open to release fluid from inflatable balloon 106 of catheter 100 to lower the pressure and volume of fluid in inflatable balloon 106 to prevent hyperinflation of inflatable balloon 106.

In the embodiment shown in FIG. 2, injection device 220 is also configured to prevent hyperinflation of inflatable balloon 106 to prevent damage to the patient's vessels. In the embodiment shown in FIG. 2, injection device 220 is electrically adjustable. Controller 206 can send a signal to injection device 220 to adjust injection device 220. Injection device 220 can be actuated to withdraw fluid from inflatable balloon 106 of catheter 100 to lower the pressure and volume of fluid in inflatable balloon 106 to prevent hyperinflation of inflatable balloon 106. In alternate embodiments, injection device 220 is not electrically adjustable.

Controller 206 is configured to receive the injected volume signal from sensing device 222. Controller 206 can determine an injected volume of fluid in inflatable balloon 106 from the injected volume signal. Controller 206 will then use the injected volume of fluid in inflatable balloon 106 to determine an actuation pressure for pressure relief valve 210. Controller determines the actuation pressure using a threshold curve, where the actuation pressure is dependent upon the inject volume of fluid in inflatable balloon 106. Once the actuation pressure has been determined, controller 206 can send a signal to pressure relief valve 210 to adjust the actuation pressure of pressure relief valve 210. In this way, the actuation pressure of pressure relief valve 210 can be dynamically adjusted based on the injected volume of fluid in inflatable balloon 106.

Controller 206 is also configured to receive the pressure signal from pressure sensor 214. Controller 206 can determine a pressure of fluid in inflatable balloon 106 from the pressure signal. Controller 206 will then use the pressure of fluid in inflatable balloon 106 to determine whether the actuation pressure, which is dependent upon the injected volume of fluid in the inflatable balloon and determined using the threshold curve, has been exceeded. If the actuation pressure has been exceeded, controller 206 can send a signal to injection device 220 to actuate injection device 220 to withdraw fluid from inflatable balloon 106. Further, controller 206 can provide a signal to a physician, such as an audible alarm, to let the physician know that the actuation pressure has been exceeded.

In the embodiment shown in FIG. 2, controller 206 will store the injected volume signals from sensing device 222 and the pressure signals from pressure sensor 214. Controller 206 can graph and store the pressure versus injected volume curve for inflatable balloon 106. The stored injected volume signals, pressure signals, and pressure versus injected volume curve can be analyzed by a physician during or after a procedure. In alternate embodiments, controller 206 will not store the injected volume signals or the pressure signals.

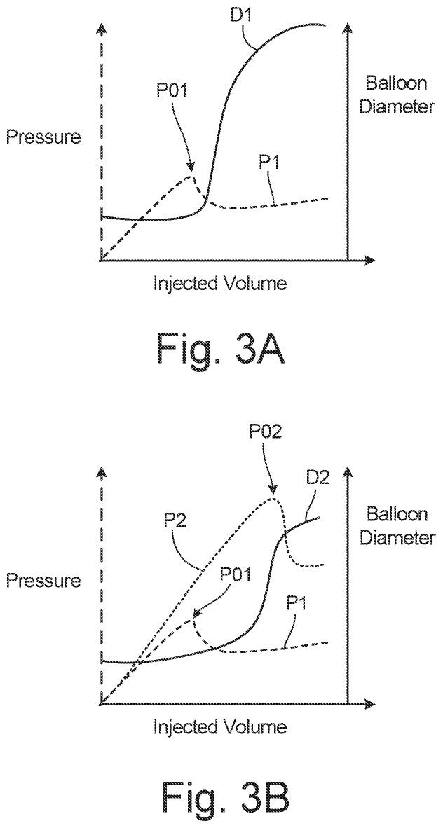

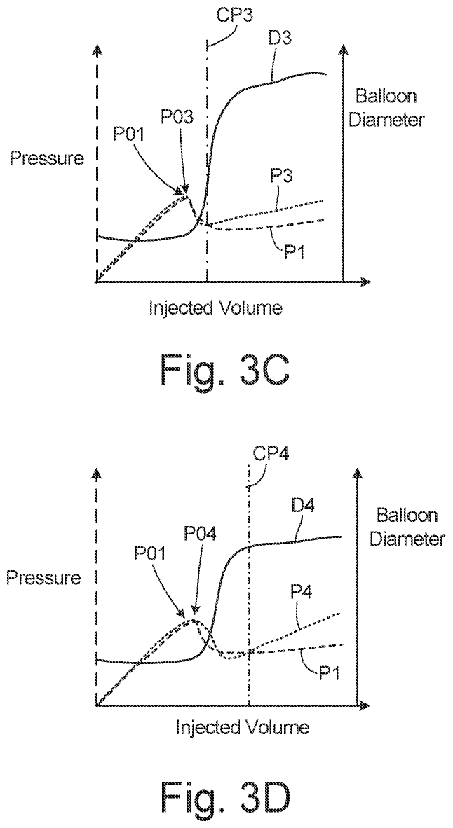

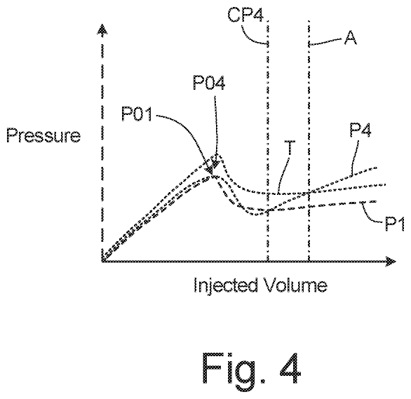

An example of a threshold curve is discussed below in reference to FIGS. 3A-4.

FIGS. 3A-3D show pressure versus injected volume curves P1, P2, P3, and P4, and balloon diameter versus injected volume curves D1, D2, D3, and D4, for an inflatable balloon. FIG. 3A is a graph showing pressure versus injected volume curve P1 and balloon diameter versus injected volume curve D1 for an inflatable balloon that is unloaded. FIG. 3B is a graph showing pressure versus injected volume curve P2 and balloon diameter versus injected volume curve D2 for the inflatable balloon in a soft, tight cylinder. FIG. 3C is a graph showing pressure versus injected volume curve P3 and balloon diameter versus injected volume curve D3 for the inflatable balloon in a soft, loose cylinder. FIG. 3D is a graph showing pressure versus injected volume curve P4 and balloon diameter versus injected volume curve D4 for the inflatable balloon in a stiff, loose cylinder. FIG. 3A shows pressure versus injected volume curve P1, pop-open point PO1, and balloon diameter versus injected volume curve D1. FIG. 3B shows pressure versus injected volume curve P1, pop-open point PO1, pressure versus injected volume curve P2, pop-open point PO2, and balloon diameter versus injected volume curve D2. FIG. 3C shows pressure versus injected volume curve P1, pop-open point PO1, pressure versus injected volume curve P3, pop-open point PO3, contact point CP3, and balloon diameter versus injected volume curve D3. FIG. 3D shows pressure versus injected volume curve P1, pop-open point PO1, pressure versus injected volume curve P4, pop-open point PO4, contact point CP4, and balloon diameter versus injected volume curve D4.

Pressure versus injected volume curves P1, P2, P3, and P4 and balloon diameter versus injected volume curves D1, D2, D3, and D4 shown in FIGS. 3A-3D are provided as examples only. Pressure versus injected volume curves P1, P2, P3, and P4 and balloon diameter versus injected volume curves D1, D2, D3, and D4 shown in FIGS. 3A-3D show differences in pressure versus injected volume curves P1, P2, P3, and P4 and differences in balloon diameter versus injected volume curves D1, D2, D3, and D4 when the same inflatable balloon is unloaded, placed in a soft, tight cylinder, placed in a soft, loose cylinder, and placed in a stiff, loose cylinder. The inflatable balloon can be inflatable balloon 106 of catheter 100 shown in and discussed in reference to FIGS. 1A-1B, or the inflatable balloon can be any other suitable inflatable balloon.

FIG. 3A shows pressure versus injected volume curve P1 and balloon diameter versus injected volume curve D1 for an inflatable balloon that is not placed in a cylinder or vessel (an unloaded inflatable balloon).

Pressure versus injected volume curve P1 depicts changes in pressure and volume of the unloaded inflatable balloon as a function of the injected volume. As a fluid is injected into the inflatable balloon, the pressure rises faster than the volume of the inflatable balloon. At a certain pressure, the inflatable balloon will pop-open. This point is depicted in FIG. 3A as pop-open point PO1. Pop-open point PO1 is the point at which the inflatable balloon, which has been compressed around the distal end of the catheter for delivery into the heart, will pop-open from its compressed state around the distal end of the catheter. After pop-open point PO1 is reached, the pressure in the inflatable balloon will decrease as the volume increases sharply. As more fluid is injected into the inflatable balloon, the pressure will rise slower than the volume of the inflatable balloon.

Balloon diameter versus injected volume curve D1 depicts changes in balloon diameter and volume for an unloaded inflatable balloon as a function of the injected volume. As shown in FIG. 3A, the balloon diameter stays relatively the same until pop-open point PO1. At pop-open point PO1, the inflatable balloon pops open and the diameter of the inflatable balloon increases quickly while the pressure in the inflatable balloon rises much slower.

FIG. 3B shows pressure versus injected volume curve P1 of the unloaded inflatable balloon, as shown in FIG. 3A, for reference. FIG. 3B also shows pressure versus injected volume curve P2 and balloon diameter versus injected volume curve D2 for an inflatable balloon that is positioned in a soft, tight cylinder (a loaded inflatable balloon). The soft, tight cylinder may resemble a human or animal blood vessel.

Pressure versus injected volume curve P2 depicts changes in pressure and volume of the loaded inflatable balloon as a function of the injected volume. When pressure versus injected volume curve P2 of the loaded inflatable balloon in a soft, tight cylinder is compared to pressure versus injected volume curve P1 of the unloaded inflatable balloon, it can be seen that for a given injected volume, the pressure of the loaded inflatable balloon is higher when compared to the unloaded balloon. This is due to the inflatable balloon being in a soft, tight cylinder. As fluid is injected into the inflatable balloon, the inflatable balloon will instantly press against the soft, tight cylinder. As a result, the pressure in the inflatable balloon will rise faster for the loaded inflatable balloon versus the unloaded inflatable balloon. Further, pop-open point PO2 for the loaded inflatable balloon occurs at a higher pressure and a higher injected volume than pop-open point PO1 for the unloaded inflatable balloon.

Balloon diameter versus injected volume curve D2 depicts changes in balloon diameter and volume of the loaded inflatable balloon as a function of the injected volume. As shown in FIG. 3B, the diameter of the inflatable balloon stays relatively small until pop-open point PO2, at which point the balloon diameter increases quickly. At pop-open point PO2, the pressure in the inflatable balloon decreases as the diameter of the inflatable balloon increases.

FIG. 3C shows pressure versus injected volume curve P1 of the unloaded inflatable balloon, as shown in FIG. 3A, for reference. FIG. 3C also shows pressure versus injected volume curve P3 and balloon diameter versus injected volume curve D3 for an inflatable balloon that is positioned in a soft, loose cylinder (the inner diameter of the cylinder is greater than the outer diameter of the inflatable balloon after it pops open at pop-open point PO3). The soft, loose cylinder may resemble a human or animal blood vessel.

Pressure versus injected volume curve P3 depicts changes in pressure and volume of the inflatable balloon in a soft, loose cylinder as a function of the injected volume. Pressure versus injected volume curve P3 mimics pressure versus injected volume curve P1 (and pop-open point PO3 for the inflatable balloon in a soft, loose cylinder is the same as the pop-open point PO1 for the unloaded inflatable balloon) until the inflatable balloon reaches the walls of the cylinder. Contact point CP3 is the point at which the inflatable balloon contacts the cylinder and is depicted by a vertical dot-dashed line in FIG. 3C. After the inflatable balloon contacts the cylinder at contact point CP3, the pressure in the inflatable balloon in the soft, loose cylinder rises faster than the pressure in the unloaded balloon. This is because the inflatable balloon begins pressing against the soft, loose cylinder after contact point CP3.

Balloon diameter versus injected volume curve D3 depicts changes in balloon diameter and volume of the inflatable balloon in a soft, loose cylinder as a function of the injected volume. As shown in FIG. 3C, the diameter of the inflatable balloon stays relatively small until pop-open point PO3, at which point the balloon diameter increases quickly. At pop-open point PO3, the pressure in the inflatable balloon decreases as the diameter of the inflatable balloon increases. Further, at contact point CP3, the rate of increase in the diameter of the inflatable balloon will slow as the pressure in the inflatable balloon rises due to the inflatable balloon pressing against the soft, loose cylinder.

FIG. 3D shows pressure versus injected volume curve P1 of the unloaded inflatable balloon, as shown in FIG. 3A, for reference. FIG. 3D also shows pressure versus injected volume curve P4 and balloon diameter versus injected volume curve D4 for an inflatable balloon that is positioned in a stiff, loose cylinder (the inner diameter of the cylinder is greater than the outer diameter of the inflatable balloon after it pops open at pop-open point PO4). The stiff, loose cylinder may resemble a human or animal blood vessel.

Pressure versus injected volume curve P4 depicts changes in pressure and volume of the inflatable balloon in a stiff, loose cylinder as a function of the injected volume. Pressure versus injected volume curve P4 mimics pressure versus injected volume curve P1 (and pop-open point PO4 for the inflatable balloon in a stiff, loose vessel mimics pop-open point PO1 for the unloaded inflatable balloon) until the inflatable balloon reaches the walls of the cylinder. Contact point CP4 is the point at which the inflatable balloon contacts the cylinder and is depicted by a vertical dot-dashed line in FIG. 3D. After the inflatable balloon contacts the cylinder at contact point CP4, the pressure in the inflatable balloon in the stiff, loose cylinder rises faster than the pressure in the unloaded balloon. This is because the inflatable balloon begins pressing against the stiff, loose cylinder after contact point CP4.

Balloon diameter versus injected volume curve D4 depicts changes in balloon diameter and volume of the inflatable balloon in a stiff, loose cylinder as a function of the injected volume. As shown in FIG. 3D, the diameter of the inflatable balloon stays relatively small until pop-open point PO4, at which point the balloon diameter increases quickly. At pop-open point PO4, the pressure in the inflatable balloon decreases as the diameter of the inflatable balloon increases. Further, at contact point CP4, the rate of increase in the diameter of the inflatable balloon will slow as the pressure in the inflatable balloon rises due to the inflatable balloon pressing against the stiff, loose cylinder.

Pressure versus injected volume curves P1, P2, P3, and P4 and the balloon diameter versus injected volume curves D1, D2, D3, and D4 can be compared to see how the pressure, diameter, and volume of the inflatable balloon varies between the unloaded inflatable balloon, the loaded inflatable balloon in a soft, tight cylinder, the inflatable balloon in a soft, loose cylinder, and the inflatable balloon in a stiff, loose cylinder. Pressure versus injected volume curve P1 can also be used to set a threshold curve that hyperinflation prevention system 200 can use to dynamically adjust pressure relief valve 210 and injection device 220 of hyperinflation prevention system 200.

FIG. 4 is a graph showing threshold curve T and pressure versus injected volume curve P4 for an inflatable balloon in a stiff, loose vessel. FIG. 4 shows pressure versus injected volume curve P1, pop-open point PO1, pressure versus injected volume curve P4, pop-open point PO4, contact point CP4, threshold curve T, and actuation point A.

Pressure versus injected volume curves P1 and P4 and threshold curve T shown in FIG. 4 are provided as examples only. Pressure versus injected volume curves P1 and P4 shown in FIG. 4 show differences in pressure versus injected volume curves P1 and P4 when the same inflatable balloon is unloaded and when the inflatable balloon is placed in a stiff, loose cylinder. The inflatable balloon can be inflatable balloon 106 of catheter 100 shown in and discussed in reference to FIGS. 1A-1B, or the inflatable balloon can be any other suitable inflatable balloon.

FIG. 4 shows pressure versus injected volume curve P1 of the unloaded inflatable balloon, as shown in and discussed in reference to FIG. 3A. FIG. 4 also shows pressure versus injected volume curve P4 for the inflatable balloon that is positioned in a stiff, loose cylinder, as shown in and discussed in reference to FIG. 3D.

FIG. 4 also shows threshold curve T. Threshold curve T mimics pressure versus injected volume curve P1 of the unloaded inflatable balloon, but threshold curve T is positively offset from pressure versus injected volume curve P1 of the unloaded inflatable balloon. This can be seen in a comparison of threshold curve T and pressure versus injected volume curve P1 of the unloaded inflatable balloon in FIG. 4. As shown in FIG. 4, threshold curve T allows for a slightly higher pressure for a given injected volume when compared to pressure versus injected volume curve P1. Threshold curve T will be different for different inflatable balloons. In some cases, the amount of offset of threshold curve T from pressure versus injected volume curve P1 is determined based on experiments. Threshold curve T can also be modified for each patient depending on the patient's age, medical history, and other such factors.

Threshold curve T can be used by hyperinflation prevention system 200 to prevent hyperinflation of an inflatable balloon. As the injected volume of fluid in the inflatable balloon changes, an actuation pressure at which fluid should be released from the inflatable balloon will change. Threshold curve T defines the relationship between the actuation pressure and the injected volume of fluid in the inflatable balloon. When the pressure in the inflatable balloon at a given injected volume crosses threshold curve T, pressure relief valve 210 of hyperinflation prevention system 200 will be actuated and/or injection device 220 of hyperinflation prevention system 200 will be actuated. Pressure versus injected volume curve P4 is shown as an example in FIG. 4. Pressure versus injected volume curve P4 crosses threshold curve T at actuation point A. Actuation point A is depicted as a vertical dot-dashed line in FIG. 4. At actuation point A, the pressure in the inflatable balloon has risen above the actuation pressure for the inflatable balloon for the injected volume of fluid in the inflatable balloon. Actuation point A is the point at which pressure relief valve 210 of hyperinflation prevention system 200 is actuated to reduce the pressure in the inflatable balloon and/or injection device 220 of hyperinflation prevention system 200 is actuated to withdraw fluid from the inflatable balloon. Actuation point A can take place at any point along threshold curve T.

The actuation pressure that is needed to actuate pressure relief valve 210 and/or injection device 220 of hyperinflation prevention system 200 is dependent upon the injected volume of fluid in the inflatable balloon. This is shown in threshold curve T, where the actuation pressure changes based on the injected volume of fluid in the inflatable balloon.

Once the pressure versus injected volume curve P1 of the unloaded balloon is known, it can be used to set the threshold curve T. If the pressure in the inflatable balloon rises above threshold curve T at any give injected volume, pressure relief valve 210 and/or injection device 220 can be actuated to reduce the pressure in the inflatable balloon to prevent hyperinflation of the inflatable balloon. Pressure relief valve 210 and injection device 220 can be dynamically adjusted by controller 206 based on an injected volume signal from sensing device 222 and a pressure signal from pressure sensor 214. The injected volume signal is a parameter sensed by the sensing device 222 that can be used to determine the injected volume of fluid in the inflatable balloon. The pressure signal is a parameter sensed by pressure sensor 214 that can be used to determine the pressure in the inflatable balloon.

Pressure sensor 214 and sensing device 222 will continue to sense the pressure in inflatable balloon and the injected volume of fluid in inflatable balloon, respectively, after pressure relief valve 210 and/or injection device 220 are actuated. When the pressure in the inflatable balloon drops below threshold curve T for a given injected volume, pressure relief valve 210 will close and/or injection device 220 will stop withdrawing fluid from the inflatable balloon. Pressure relief valve 210 can be automatically closed with a spring in some embodiments.

Prior art systems aimed at preventing hyperinflation of inflatable balloons set an actuation pressure that is independent of the injected volume of fluid in the inflatable balloon. These prior art systems only sense the pressure within the inflatable balloon to determine whether that actuation pressure has been exceeded to actuate a pressure relief valve. Only taking the pressure inside the inflatable balloon into account disregards the pressure the inflatable balloon exerts on the artery wall.

Hyperinflation prevention system 200 is configured so that the maximum pressure a balloon is allowed to operate in is defined as a function of the injected volume. As a result, the inflatable balloon can safely operate in vessels of different diameters. Preventing hyperinflation of the inflatable balloon prevents dangerous complications that can arise when using pulmonary artery catheters. For example, preventing hyperinflation of the inflatable balloon can prevent damage to the blood vessels, including pulmonary artery aneurysm or rupture.

Hyperinflation prevention system 200 can also be used to detect the contact point of the inflatable balloon with the vessel and the diameter of the vessel. The contact point of the inflatable balloon can be detected at the point at which the pressure versus injected volume curve for the inflatable balloon in a cylinder deviates from pressure versus injected volume curve P1 for the unloaded inflatable balloon. For example, in FIGS. 3C-3D, contact points CP3 and CP4 are the points at which pressure versus injected volume curve P3 and pressure versus injected volume curve P4, respectively, deviate from pressure versus injected volume curve P1 of the unloaded inflatable balloon. In FIG. 3B, the inflatable balloon is in contact with the cylinder when the inflatable balloon begins to be inflated, which is indicated by pressure versus injected volume curve P2 deviating from pressure versus injected volume curve P1 as soon as fluid is injected into the inflatable balloon. Once the contact point with the vessel has been identified, the diameter of the vessel can be determined based on the properties of the balloon and the contact point with the vessel.

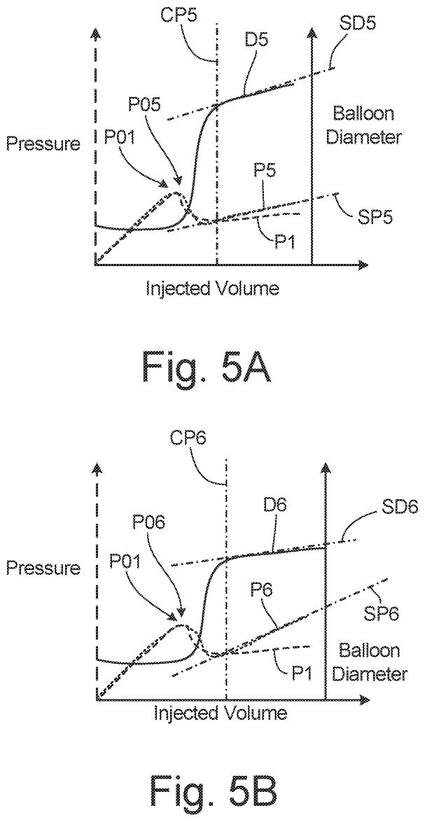

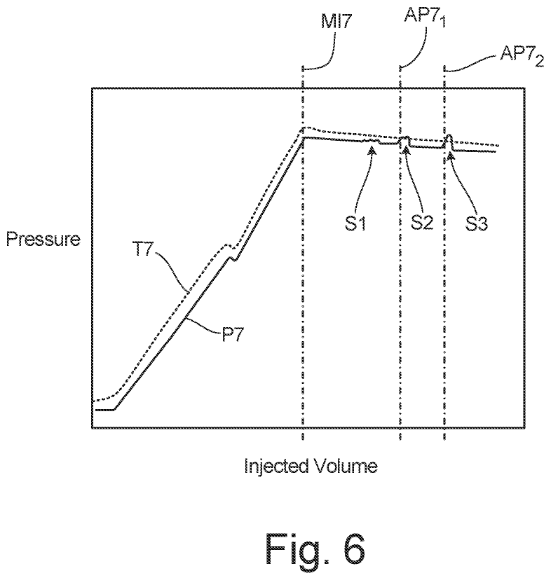

Additionally, hyperinflation prevention system 200 can also be used to determine a compliance (elasticity) of a patient's vessel, which can have diagnostic value (discussed in greater detail with respect to FIGS. 5A-5B). Further, hyperinflation prevention system 200 can reduce the pressure in the inflatable balloon when the catheter is pulled and the inflatable balloon is still inflated (discussed in greater detail with respect to FIG. 6).

FIGS. 5A-5B show pressure versus injected volume curves and balloon diameter versus injected volume curves for an inflatable balloon. FIG. 5A is a graph showing pressure versus injected volume curve P5 and balloon diameter versus injected volume curve D5 for an inflatable balloon in a soft, loose vessel. FIG. 5B is a graph showing pressure versus injected volume curve P6 and balloon diameter versus injected volume curve D6 for an inflatable balloon in a stiff, loose vessel. FIG. 5A shows pressure versus injected volume curve P1, pop-open point PO1, pressure versus injected volume curve P5, pop-open point PO5, contact point CP5, balloon diameter versus injected volume curve D5, slope SP5, and slope SD5. FIG. 5B shows pressure versus injected volume curve P1, pop-open point PO1, pressure versus injected volume curve P6, pop-open point PO6, contact point CP6, balloon diameter versus injected volume curve D6, slope SP6, and slope SD6.

The pressure versus injected volume curves and the balloon diameter versus injected volume curves shown in FIGS. 5A-5B are provided as examples only. The pressure versus injected volume curves a shown in FIGS. 5A-5B show differences in pressure versus injected volume curves when the same inflatable balloon is placed in a soft, loose vessel and a stiff, loose vessel. The inflatable balloon can be inflatable balloon 106 of catheter 100 shown in and discussed in reference to FIGS. 1A-1B, or the inflatable balloon can be any other suitable inflatable balloon. FIGS. 5A-5B show pressure versus injected volume curve P1 of the unloaded inflatable balloon, as shown in FIG. 3A, for reference.

FIG. 5A shows pressure versus injected volume curve P5 and balloon diameter versus injected volume curve D5 for an inflatable balloon in a soft, loose vessel (the inner diameter of the vessel is larger than the outer diameter of the inflatable balloon after pop-open point PO5). As shown in FIG. 5A, pressure versus injected volume curve P5 mimics pressure versus injected volume curve P1 until contact point CP5. Pop-open point PO5 of the inflatable balloon in the soft, loose vessel happens at about the same point as pop-open point PO1 of the unloaded inflatable balloon. At pop-open point PO5, the diameter of the inflatable balloon will rise quickly, as shown by balloon diameter versus injected volume curve D5. As the diameter of the inflatable balloon rises quickly, the pressure in the inflatable balloon will drop as shown with pressure versus injected volume curve P5. The inflatable balloon will contact the vessel at contact point CP5 (shown by a vertical dot-dashed line in FIG. 5A). At contact point CP5, the diameter of the inflatable balloon will slowly rise as the inflatable balloon presses against the vessel and the pressure in the inflatable balloon will rise.

FIG. 5B shows pressure versus injected volume curve P6 and balloon diameter versus injected volume curve D6 for an inflatable balloon in a stiff, loose vessel (the inner diameter of the vessel is larger than the outer diameter of the inflatable balloon after pop-open point P)6). As shown in FIG. 5B, pressure versus injected volume curve P6 mimics pressure versus injected volume curve P1 until contact point CP6. Pop-open point PO6 of the inflatable balloon in the stiff, loose vessel happens at about the same point at pop-open point PO1 of the unloaded inflatable balloon. At pop-open point PO6, the diameter of the inflatable balloon will rise quickly, as shown by balloon diameter versus injected volume curve D6. As the diameter of the inflatable balloon rises quickly, the pressure in the inflatable balloon will drop as shown with pressure versus injected volume curve PO6. The inflatable balloon will contact the vessel at contact point CP6 (shown by a vertical dot-dashed line in FIG. 5B). At contact point CP6, the diameter of the inflatable balloon will slowly rise as the inflatable balloon presses against the vessel and the pressure in the inflatable balloon will rise.

FIG. 5A shows slope SP5 and slope SD5, which are shown by dot-dashed lines in FIG. 5A. Slope SP5 is the slope of pressure versus injected volume curve P5 after contact point CP5. Slope SD5 is the slope of balloon diameter versus injected volume curve D5 after contact point CP5. FIG. 5B shows slope SP6 and slope SD6, which are shown by dot-dashed lines in FIG. 5B. Slope SP6 is the slope of pressure versus injected volume curve P6 after contact point CP6. Slope SD6 is the slope of balloon diameter versus injected volume curve D6 after contact point CP6.

Slope SP5 and slope SD5 of the inflatable balloon in the soft vessel and slope SP6 and slope SD6 of the inflatable balloon in the stiff vessel can be compared to determine the differences in the compliance of the vessels. As shown in FIG. 5A, slope SD5 of the diameter of the inflatable balloon in a soft vessel is greater than slope SD6 of the diameter of the inflatable balloon in a stiff vessel. In a soft vessel, as more fluid is injected into the inflatable balloon it will press against the walls and cause the walls of the soft vessel to expand. This is shown by the continued increase in the balloon diameter after the inflatable balloon comes into contact with the vessel at contact point CP5. The pressure in the inflatable balloon in the soft vessel will increase more slowly as the diameter of the inflatable balloon continues to increase. In a stiff vessel, as more fluid is injected into the inflatable balloon it will press against the walls of the stiff vessel, but the stiff vessel will not expand as quickly as the walls of the soft vessel. This is shown by the smaller increase in the balloon diameter of the inflatable balloon in the stiff vessel after the inflatable balloon comes into contact with the vessel at contact point CP6 when compared to the increase in the balloon diameter of the inflatable balloon in the soft vessel. The pressure in the inflatable balloon in the stiff vessel will increase more quickly as the diameter of the inflatable balloon increases more slowly.

Comparing slope SP5 and slope SD5 of the inflatable balloon in the soft vessel and slope SP6 and slope SD6 of the inflatable balloon in the stiff vessel can indicate the compliance (or elasticity) of the vessel in which the inflatable balloon is positioned. As shown in FIGS. 5A-5B, after the inflatable balloon comes into contact with the vessel, the balloon diameter will rise faster in a soft vessel than in a stiff vessel. As further shown in FIGS. 5A-5B, after the inflatable balloon comes into contact with the vessel, the pressure will rise faster in a stiff vessel that in a soft vessel. As such, the slope of pressure versus injected volume curves and balloon diameter versus injected volume curves after an inflatable balloon comes into contact with a vessel can indicate the compliance (or elasticity) of the vessel. Measuring and monitoring the elasticity of vessels has diagnostic value. For example, the elasticity of vessels can help when titrating vasoactive drugs to treat pulmonary hypertension.

FIG. 6 is a graph showing threshold curve T7 and pressure versus injected volume curve P7 for an inflatable balloon that is being pulled while inflated. FIG. 6 shows pressure versus injected volume curve P7, threshold curve T7, maximum inflation MI7, spike S1, spike S2, spike S3, actuation point AP71, and actuation point AP72.

FIG. 6 shows pressure versus injected volume curve P7 for an inflatable balloon. Threshold curve T7 is also shown. As discussed above in reference to FIG. 4, threshold curve T7 sets an actuation pressure that is dependent upon the injected volume of fluid in the inflatable balloon.

Maximum inflation MI7 is shown in FIG. 6 as a vertical dot-dashed line. Maximum inflation MI7 is the maximum volume of fluid that is to be injected into the inflatable balloon. At maximum inflation MI7, hemodynamic parameters can be determined. Once the hemodynamic parameters are determined, the inflatable balloon should be deflated before the catheter is removed from the patient. However, in some instances, physicians may forget to deflate the inflatable balloon before removing it from the patient or the inflatable balloon may not properly deflate. Pulling an inflated balloon can cause serious damage to a patient's vessels.

Hyperinflation prevention system 200 can be used to prevent inflatable balloons from being pulled before being deflated to prevent damage to the patient's vessels. There are three spikes S1, S2, and S3 in pressure shown in FIG. 6. Spikes S1, S2, and S3 show pressure spikes that happen when an inflatable balloon is pulled by a physician before being deflated. When a physician pulls an inflatable balloon that is still inflated, the deformation of the inflatable balloon will cause a spike in pressure in the inflatable balloon. Spike S1 does not cross threshold curve T7, so neither pressure relief valve 210 nor injection device 220 of hyperinflation prevention system 200 are actuated. Spikes S2 and S3, however, do cross threshold curve T7. As the pressure rises above threshold curve T7, pressure relief valve 210 and/or injection device 220 of hyperinflation prevention system 200 will be actuated to reduce the pressure in the inflatable balloon. Actuation point AP71 and actuation point AP72 are shown as vertical dot-dashed lines in FIG. 6 and indicate the point whether the pressure rises above threshold curve T7 and where pressure relief valve 210 and/or injection device 220 will be actuated.

Actuation of pressure relief valve 210 and/or injection device 220 as a catheter is pulled when the inflatable balloon is still inflated can prevent damage to the patient's vessels. Controller 206 of hyperinflation prevention system 200 may also provide a signal, such as an audible alarm, to a physician when threshold curve T7 is exceeded, which would indicate that the inflatable balloon has not been deflated and that the catheter should not be pulled. Further, controller 206 may include a screen upon which pressure versus injected volume curve P7 is graphed. A physician monitoring pressure versus injected volume curve P7 can see spikes S1, S2, and S3 that indicate the inflatable balloon is still inflated, which would indicate that the catheter should not be pulled until the inflatable balloon is deflated.

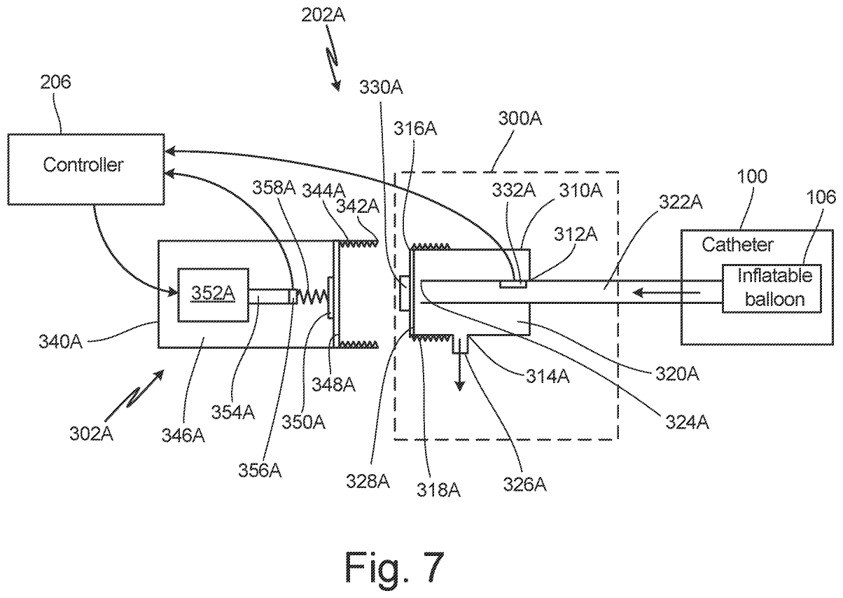

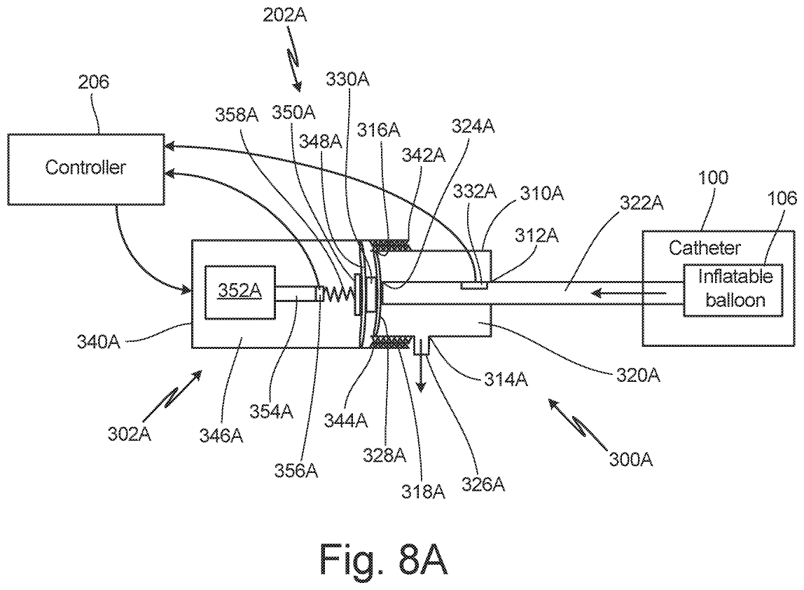

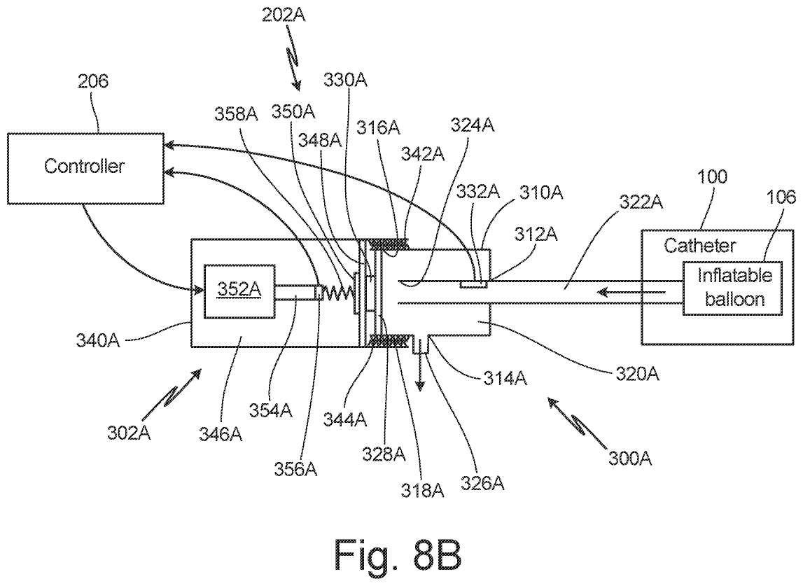

FIG. 7 is a partial cross-sectional view of valve mechanism 202A. FIG. 8A is a partial cross-sectional view of valve mechanism 202A in a closed position. FIG. 8B is a partial cross-sectional view of valve mechanism 202A in an open position. FIGS. 7-8B shows catheter 100, inflatable balloon 106, valve mechanism 202A, and controller 206. Valve mechanism 202A includes disposable part 300A and reusable part 302A. Disposable part 300A includes body 310A, opening 312A, opening 314A, opening 316A, threads 318A, cavity 320A, tube 322A, opening 324A, outlet 326A, disk seal 328A, ferromagnetic contact 330A, and pressure sensor 332A. Reusable part 302A includes body 340A, opening 342A, threads 344A, cavity 346A, disk seal 348A, magnet 350A, linear actuator 352A, shaft 354A, force sensor 356A, and spring 358A.

Valve mechanism 202A is one embodiment of a valve mechanism that can be used in hyperinflation prevention system 200 (shown in FIG. 2). Valve mechanism 202A is fluidly coupled to catheter 100 in the embodiment shown in FIGS. 7-8B. Valve mechanism 202A can be fluidly coupled to any suitable catheter in alternate embodiments. Valve mechanism 202A is coupled by a wired or wireless communication link to controller 206 of hyperinflation prevention system 200. Valve mechanism 202A can send signals to and receive signals from controller 206. Valve mechanism 202A is a single, integral unit that acts as a pressure relief valve, a check valve, and a pressure sensor, such as pressure relief valve 210, check valve 212, and pressure sensor 214 of hyperinflation prevention system 200 (shown in FIG. 2).

Valve mechanism 202A includes disposable part 300A and reusable part 302A. Disposable part 300A is configured to be a single use product and should be disposed after use. Disposable part 300A is designed to be simple and cheap to manufacture. Reusable part 302A is configured to be reused any number of times. Disposable part 300A and reusable part 302A are configured to be releasably connected to one another.

Disposable part 300A includes body 310A that forms a housing for disposable part 300A. Body 310A is cylindrically shaped in the embodiment shown in FIGS. 7-8B, but can be any suitable shape in alternate embodiments. Body 310A includes opening 312A at a first end of body 310A, opening 314A positioned on an annular wall of body 310A between the first end and a second end, and opening 316A at the second end of body 310A. Body 310A also includes threads 318A positioned on an exterior of the annular wall of body 310A adjacent the second end of body 310A. Threads 318A are configured to attach disposable part 300A to reusable part 302A. Cavity 320A is formed in body 310A of disposable part 300A.

Disposable part 300A further includes tube 322A that extends through and is sealed in opening 312A on the first end of body 310A and through cavity 320A of body 310A. A first end of tube 322A is fluidly coupled to catheter 100. A second end of tube 322A is positioned adjacent to the second end of body 310A. Tube 322A includes opening 324A at the second end. Tube 322A is an inlet to cavity 320A of body 310A.

Disposable part 300A also includes outlet 326A that is positioned at opening 314A of body 310A. In the embodiment shown in FIGS. 7-8B, outlet 326A is open to an ambient around disposable part 300A. Disk seal 328A is positioned in and sealed in opening 316A at the second end of body 310A. Ferromagnetic contact 330A is positioned on disk seal 328A outside of cavity 320A of body 310A. Pressure sensor 332A is positioned in tube 322A and is configured to sense a pressure in tube 322A. Pressure sensor 332A is coupled by a wired or wireless communication link to controller 206.

Reusable part 302A includes body 340A that forms a housing for reusable part 302A. Body 340A is cylindrically shaped in the embodiment shown in FIGS. 7-8B, but can be any suitable shape in alternate embodiments. Body 340A includes opening 342A at a first end of body 340A. Body 340A also includes threads 344A positioned on an interior of the annular wall of body 340A adjacent the first end of body 340A. Threads 344A are configured to attached reusable part 302A to disposable part 300A. Cavity 346A is formed in body 340A of reusable part 302A.

Reusable part 302A further includes disk seal 348A positioned in and sealed in opening 342A adjacent the first end of body 340A. Disk seal 348A is set back into opening 342A behind threads 344A so that threads 344A are accessible. Magnet 350A is positioned on disk seal 348A in cavity 346A of body 340A. Reusable part 302A also includes linear actuator 352A positioned in cavity 346A of body 340A. Linear actuator 352A includes shaft 354A with force sensor 356A at an end of shaft 354A. Linear actuator 352A and force sensor 356A are coupled by a wired or wireless communication link to controller 206. Spring 358A extends between force sensor 356A and magnet 350A.

Disposable part 300A and reusable part 302A are connected to one another with threads 318A on body 310A of disposable part 300A and threads 344A on body 340A of reusable part 302A, as shown in FIGS. 8A-8B. In alternate embodiments, disposable part 300A and reusable part 302A can be connected using any suitable means, for example a luer lock. When disposable part 300A and reusable part 302A are connected to one another, disk seal 328A acts as a protective seal and isolates the fluid in tube 322A and cavity 320A of body 310A to prevent the fluid from entering reusable part 302A. Disk seal 348A acts as a secondary protective seal to also isolate fluid from entering cavity 346A of body 340A.

Magnet 350A will magnetically connect to ferromagnetic contact 330A when reusable part 302A and disposable part 300A of valve mechanism 202A are connected. As disk seal 328A moves between a deformed and undeformed state, disk seal 348A will also move between the deformed and undeformed state due to the magnetic coupling between magnet 350A and ferromagnetic contact 330A. Similarity, if disk seal 348A moves between a deformed and undeformed state, disk seal 328A will also move between the deformed and undeformed state due to the magnetic coupling between magnet 350A and ferromagnetic contact 330A.

FIG. 8A shows valve mechanism 202A in a closed position. Valve mechanism 202B will be in the closed position when a pressure in catheter 100 is below an actuation pressure of the valve mechanism. As shown in FIG. 8A, when valve mechanism 202A is in the closed position, disk seal 328A and disk seal 348A are both deformed. Disk seal 328A and disk seal 348A are deformed due to a load of spring 358A that applies a force to magnet 350A and disk seal 348A of reusable part 302A, and thus to ferromagnetic contact 330A and disk seal 328A of disposable part 300A. When disk seal 328A of disposable part 300A is deformed, it will seal against opening 324A of tube 322A. This will close tube 322A and prevent fluid in tube 322A from flowing from tube 322A to cavity 320A of body 310A and out of outlet 326A.

The load being placed on spring 358A determines an actuation pressure that is needed to actuate valve mechanism 202A. FIG. 8B shows valve mechanism 202A in an open position. Valve mechanism 202A will move to the open position when the pressure in catheter 100 exceeds the actuation pressure, which causes actuation of valve mechanism 202A. In the open position, the pressure of the fluid in catheter 100 presses against disk seal 328A and overcomes the force being placed on disk seal 328A due to the load of spring 358A. When the disk seal 328A is pressed to its undeformed state due to the pressure in catheter 100, disk seal 348A will also be pressed to its undeformed state due to magnet 350A and ferromagnetic contact 330A. In the open position, the fluid in tube 322A can flow through opening 324A of tube 322A, into cavity 320A of body 310A, and out of body 310A through outlet 326A. This will reduce the pressure in catheter 100 to prevent hyperinflation of inflatable balloon 106 of catheter 100.

After the pressure in catheter 100 has been relieved, spring 358A will automatically close valve mechanism 202A. As the pressure in catheter 100 drops, the load of spring 358A will push against magnet 350A, causing disk seal 348A and disk seal 328A to deform. When disk seal 328A is deformed, it will cover opening 324A of tube 322A to isolate the fluid in catheter 100.

Linear actuator 352A puts the load on spring 358A. Linear actuator 352A is coupled by a wired or wireless communication link to and can be controlled by controller 206. Controller 206 can send a signal to linear actuator 352A to vary the load that is being placed on spring 358A. Varying the load that is placed on spring 358A can vary the actuation pressure that is needed to actuate valve mechanism 202A to relieve pressure in catheter 100, as the force being placed upon disk seal 328A will change. Linear actuator 352A can vary the load on spring 358A with extension and retraction of shaft 354A, which varies the extension and retraction, and thus the load, of spring 358A.

Force sensor 356A is positioned between shaft 354A and spring 358A to sense the load on spring 358A. Force sensor 356A is coupled by a wired or wireless communication link to controller 206 and can send a force signal to controller 206 that represents the load placed on spring 358A. Force sensor 356A is used as a redundant safety measure to ensure the load on spring 358A does not exceed a set threshold. In alternate embodiments, force sensor 356A can be a strain-gauge sensor coupled or attached to a deformable material of known mechanical properties.

Valve mechanism 202A also acts as a check valve. If pressure sensor 332A senses a negative pressure in catheter 100, it can send a signal to controller 206. Controller 206 can then send a signal to linear actuator 352A to actuate valve mechanism 202A to prevent negative pressure in catheter 100. Linear actuator 352A will retract shaft 354A, which will in turn pull force sensor 356A, spring 358A, magnet 350A, and disk seal 348A towards linear actuator 352A. As magnet 350A is pulled, it will pull ferromagnetic contact 330A and disk seal 328A to the open position and away from opening 324A of tube 322A. This will allow fluid in the ambient surrounding catheter 100 to flow through outlet 326A and cavity 320A into catheter 100 through opening 324A of tube 322A.

Valve mechanism 202A is an electrically adjustable pressure relief valve that can be used in hyperinflation prevention system 200 (shown in FIG. 2). As the injected volume of fluid in inflatable balloon 106 changes, the actuation pressure needed to actuate valve mechanism 202A will vary according to a threshold pressure versus injected volume curve (an example of which is shown in FIG. 4). Controller 206 will receive an injected volume signal indicating the injected volume of fluid in inflatable balloon 106. Controller 206 will then determine an actuation pressure that is dependent on the injected volume of fluid in inflatable balloon 106. Controller 206 will then send a signal to linear actuator 352A to vary the load being placed on spring 358A, which will thus vary the force being placed on disk seal 348A and disk seal 328A. As such, the actuation pressure that is needed to actuate valve mechanism 202A is dynamically adjusted based on the injected volume of fluid in inflatable balloon 106.

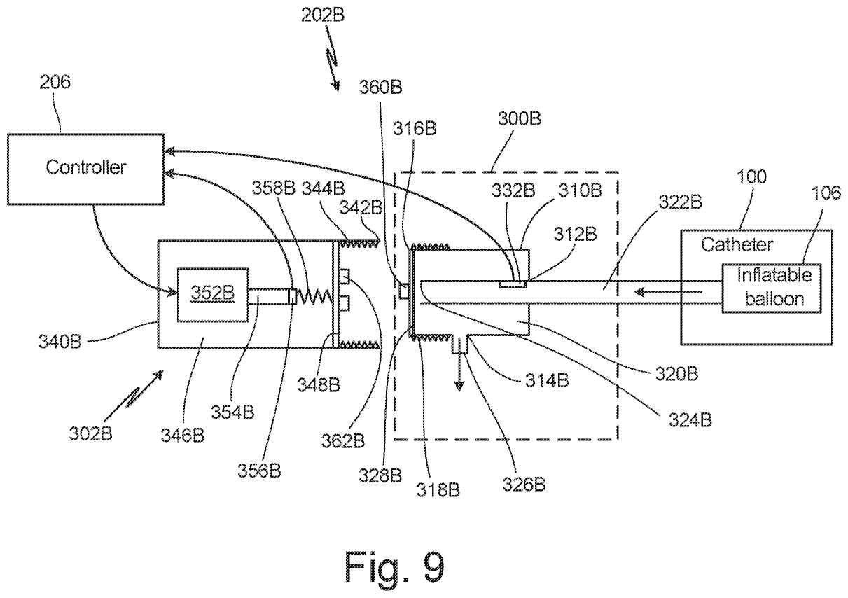

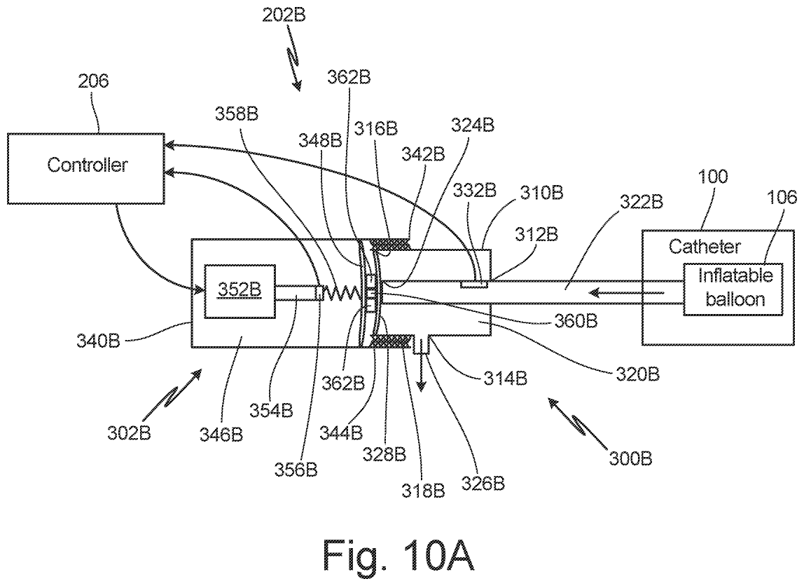

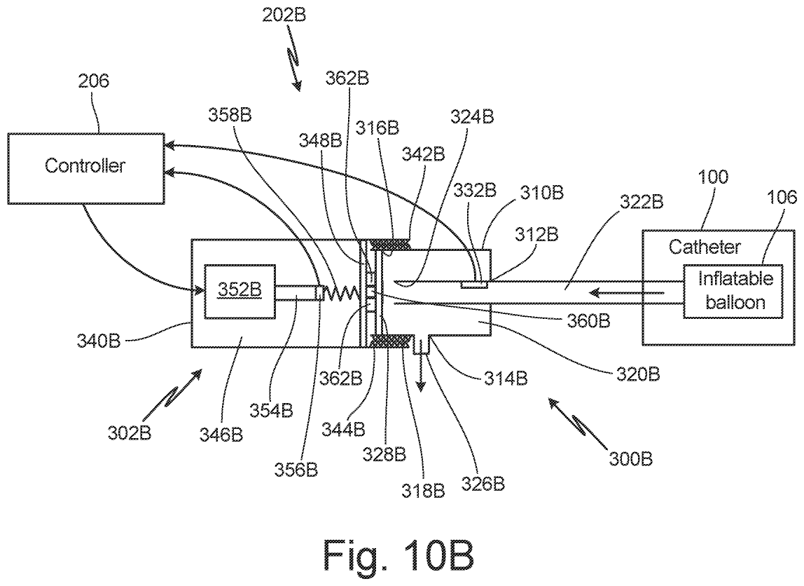

FIG. 9 is a partial cross-sectional view of valve mechanism 202B. FIG. 10A is a partial cross-sectional view of valve mechanism 202B in a closed position. FIG. 10B is a partial cross-sectional view of valve mechanism 202B in an open position. FIGS. 9-10B show catheter 100, inflatable balloon 106, valve mechanism 202B, and controller 206. Valve mechanism 202B includes disposable part 300B and reusable part 302B. Disposable part 300B includes body 310B, opening 312B, opening 314B, opening 316B, threads 318B, cavity 320B, tube 322B, opening 324B, outlet 326B, disk seal 328B, mechanical coupling 360B, and pressure sensor 332B. Reusable part 302B includes body 340B, opening 342B, threads 344B, cavity 346B, disk seal 348B, mechanical coupling 362B, linear actuator 352B, shaft 354B, force sensor 356B, and spring 358B.

Valve mechanism 202B is a second embodiment of a valve mechanism that can be used in hyperinflation prevention system 200 (shown in FIG. 2). Valve mechanism 202B is fluidly coupled to catheter 100 in the embodiment shown in FIGS. 9-10B. Valve mechanism 202B can be fluidly coupled to any suitable catheter in alternate embodiments. Valve mechanism 202B is coupled by a wired or wireless communication link to controller 206 of hyperinflation prevention system 200. Valve mechanism 202B can send signals to and receive signals from controller 206. Valve mechanism 202B is a single, integral unit that acts as a pressure relief valve, a check valve, and a pressure sensor, such as pressure relief valve 210, check valve 212, and pressure sensor 214 of hyperinflation prevention system 200 (shown in FIG. 2).

Valve mechanism 202B has generally the same structure and design of valve mechanism 202A shown in FIGS. 7-8B. However, valve mechanism 202B does not include a magnetic coupling between disposable part 300B and reusable part 302B. Rather, valve mechanism 202B includes a mechanical coupling between disposable part 300B and reusable part 302B. Disposable part 300B includes mechanical coupling 360B positioned on disk seal 328B outside of cavity 320B. Reusable part 302B includes mechanical coupling 362 positioned on disk seal 328B outside of cavity 346B. Mechanical coupling 360B and mechanical coupling 362 can be threaded connectors, snap connectors, clip connectors, bayonet connectors, or any other suitable mechanical connectors. Spring 358B extends between force sensor 356B and disk seal 328B.

Mechanical coupling 360B and mechanical coupling 362B can be connected using any suitable mechanism. Mechanical coupling 360B and mechanical coupling 362B may be preferable to a magnetic coupling when valve mechanism 202B is used in the presence of large magnetic fields, for example rooms containing MRI equipment.

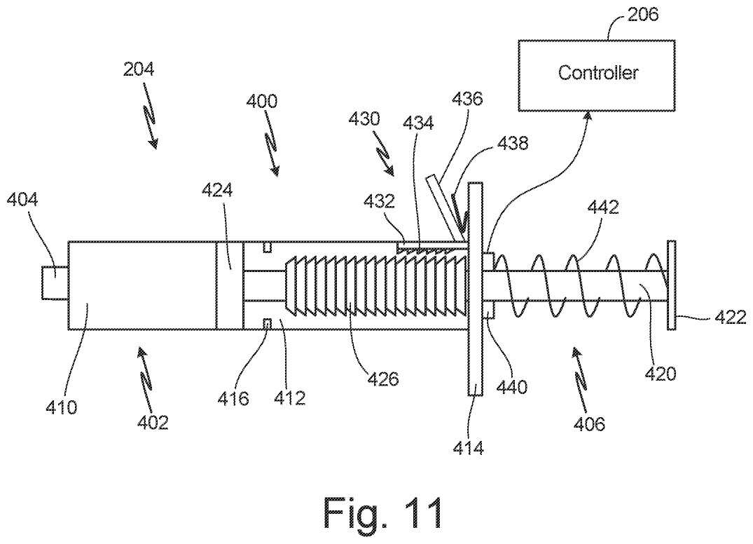

FIG. 11 is a side view of delivery mechanism 204. FIG. 11 shows delivery mechanism 204 and controller 206. Delivery mechanism 204 includes syringe 400 that includes barrel 402, tip 404, and plunger 406. Barrel 402 includes body 410, cavity 412, barrel flange 414, and displacement limiter 416. Plunger 406 includes plunger rod 420, plunger flange 422, plunger head 424, and teeth 426. Delivery mechanism 204 further includes locking mechanism 430 that includes body 432, teeth 434, trigger 436, and spring 438. Delivery mechanism 204 also includes volume displacement sensor 440 and spring 442.

Delivery mechanism 204 is one embodiment of a delivery mechanism that can be used in hyperinflation prevention system 200 (shown in FIG. 2). Delivery mechanism 204 can be fluidly coupled to catheter 100 in the embodiment shown in FIG. 11. Delivery mechanism 204 can be fluidly coupled to any suitable catheter in alternate embodiments. Delivery mechanism 204 is coupled by a wired or wireless communication link to controller 206 of hyperinflation prevention system 200. Delivery mechanism 204 can send signals to controller 206. Delivery mechanism 204 is a single, integral unit that acts as an injection device and a sensing device, such as injection device 220 and sensing device 222 of hyperinflation prevention system 200 (shown in FIG. 2).

Delivery mechanism 204 includes syringe 400 that is configured to inject a fluid into the catheter. Syringe 400 includes barrel 402, tip 404, and plunger 406. Barrel 402 includes body 410 that forms a housing portion for barrel 402. Cavity 412 is formed in body 410 of barrel 402 and is configured to contain the fluid that is to be injected into the catheter. Barrel 402 further includes barrel flange 414 positioned on a first end of body 410 that is configured to be grasped by a physician or other user when using syringe 400. Tip 404 is positioned at a second end of body 410 of barrel 402. Tip 404 is used to connect syringe 400 to a catheter. Tip 404 can be connected to the catheter using any suitable connection mechanism, for example a luer lock. Barrel 402 also includes displacement limiter 416 positioned in cavity 412 of syringe 400. Displacement limiter 416 is a ring-shaped stop that is attached to an interior surface of body 410 of barrel 402. Displacement limiter 416 is configured to prevent plunger 406 from being displaced beyond a predetermined distance.

Plunger 406 of syringe 400 includes plunger rod 420. Plunger flange 422 is positioned on a first end of plunger rod 420 and is configured to be grasped by a physician or other user when using syringe 400. Plunger head 424 is positioned at a second end of plunger rod 420. Plunger head 424 is sized to fit tightly in cavity 412 of barrel 402 to form a seal between plunger head 424 and body 410 of barrel 402. Plunger 406 also includes teeth 426 positioned around plunger rod 420 in cavity 412 of barrel 402.

Delivery mechanism 204 also includes locking mechanism 430 that is configured to lock plunger 406 of syringe 400 in position in barrel 402 of syringe 400. Locking mechanism 430 includes body 432 positioned in cavity 412 of body 410 of barrel 402. Teeth 434 are positioned on body 432 of locking mechanism 430 and are configured to engage teeth 426 on plunger rod 420 of plunger 406. Trigger 436 of locking mechanism 430 extends through body 410 of barrel 402 and is connected to body 432 locking mechanism 430. Spring 438 of locking mechanism 430 is positioned between trigger 436 and barrel flange 414. Locking mechanism 430 can be engaged by pressing trigger 436. When locking mechanism 430 is engaged, teeth 434 of locking mechanism 430 are pressed inwards to engage teeth 426 of plunger 406 to lock plunger 406 in position in barrel 402. To disengage locking mechanism 430, trigger 436 is pressed to overcome the spring bias of spring 438 to release locking mechanism 430 to disengage teeth 434 of locking mechanism 430 from teeth 426 on plunger 406. When locking mechanism 430 is disengaged, inflatable balloon can automatically deflate. Teeth 426 extend around plunger rod 420 of plunger 406 so that plunger rod 420 of plunger 406 has cylindrical symmetry in the area adjacent to locking mechanism 430. Locking mechanism 430 can engage teeth 426 on plunger rod 420 of plunger 406 regardless of the rotation of plunger 406.

Locking mechanism 430 is positioned under barrel flange 414 for case of use with just one finger. That allows syringe 400 and locking mechanism 430 to be used with a single hand, as a physician or other user can press on plunger 406 to inject fluid into the inflatable balloon and press trigger 436 to lock plunger 406 into position using a single hand. In comparison with the prior art (where a syringe is used together with a one-way valve to stabilize the injected volume inside catheter balloon using two hands), locking mechanism 430 does not require a one-way valve and can be operated with only one hand. Single-hand operation is valuable in clinical setups.

Delivery mechanism 204 also includes volume displacement sensor 440 that is positioned around plunger rod 420 of plunger 406 and on barrel flange 414. Spring 442 is positioned around plunger rod 420 of plunger 406 and extends between volume displacement sensor 440 and plunger flange 422. Spring 442 provides for automatic retraction of plunger 406. In alternate embodiments, volume displacement sensor 440 can be positioned on barrel flange 414 and adjacent to plunger rod 420 of plunger 406.

In the present embodiment, volume displacement sensor 440 is a force sensor. When plunger 406 is pressed into barrel 402, spring 442 will compress and the load of spring 442 will apply a force onto volume displacement sensor 440. Volume displacement sensor 440 is coupled by a wired or wireless communication link to controller 206. Volume displacement sensor 440 will sense the force that is being applied to volume displacement sensor 440 by spring 442 and send a signal of the sensed force to controller 206. Controller 206 can then determine an injected volume of fluid in an inflatable balloon of the catheter based on the force, as the force that spring 442 applies on volume displacement sensor 440 is a function of the position of plunger 406 per Hooke's law, and therefore of the volume of fluid that has been injected into catheter 100. The signal of the sensed force sent from volume displacement sensor 440 to controller 206 is the injected volume signal, as discussed in reference to FIGS. 2-4. In alternate embodiments, volume displacement sensor 440 can be any sensor that is capable of determining a displacement of plunger 406, including a strain gauge sensor, an optical encoder, a magnetic encoder, or a capacitive encoder.

Pulmonary artery catheters do not include a device for measuring the volume of fluid injected into the inflatable balloon used to obtain the wedge pressure. As a result, the inflatable balloon can be hyperinflated and damage the blood vessel. Delivery mechanism 204 includes volume displacement sensor 440 to sense a signal that indicates the amount of fluid injected into the inflatable balloon. Delivery mechanism 204 can thus be used in hyperinflation prevention system 200 to prevent hyperinflation of inflatable balloon 106 (shown in FIG. 2).

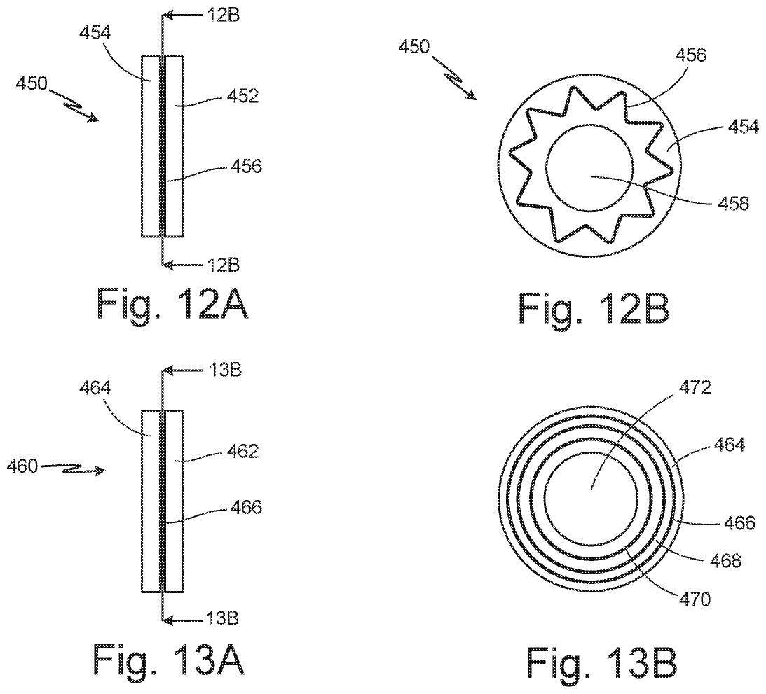

FIG. 12A is a side view of strain gauge sensor 450. FIG. 12B is a cross-sectional view of strain gauge sensor 450, taken along line 12B-12B of FIG. 12A. Strain gauge sensor 450 includes first body portion 452, second body portion 454, wire 456, and opening 458.

First body portion 452 and second body portion 454 of strain gauge sensor 450 are ring shaped. First body portion 452 and second body portion 454 are made out a compliant material. First body portion 452 and second body portion 454 are positioned on top of one another and wire 456 is sandwiched between first body portion 452 and second body portion 454. Wire 456 can be a conductive track or a trace in alternate embodiments. Wire 456 has a wavy shape and is positioned in a circle between first body portion 452 and second body portion 454. Opening 458 extends through a center of first body portion 452 and second body portion 454.

Strain gauge sensor 450 is a first example of a volume displacement sensor that can be used on delivery mechanism 204 (shown in FIG. 11). Opening 458 can be positioned around plunger rod 420 of plunger 406 and strain gauge sensor 450 can be positioned against barrel flange 414 of barrel 402. Strain gauge sensor 450 will thus act as volume displacement sensor 440 and can sense the force placed on it by the compression of spring 442 of delivery mechanism 204. A volume of fluid that has been injected into the inflatable balloon can be determined from the force sensed by strain gauge sensor 450 using Hooke's law.

FIG. 13A is a side view of strain gauge sensor 460. FIG. 13B is a cross-sectional view of strain gauge sensor 460, taken along line 13B-13B of FIG. 13A. Strain gauge sensor 460 includes first body portion 462, second body portion 464, wire 466, wire 468, wire 470, and opening 472.

First body portion 462 and second body portion 464 of strain gauge sensor 460 that are ring shaped. First body portion 462 and second body portion 464 are made out a compliant material. First body portion 462 and second body portion 464 are positioned on top of one another and wire 466, wire 468, and wire 470 are sandwiched between first body portion 462 and second body portion 464. Wire 466, wire 468, and wire 470 can be conductive tracks or traces in alternate embodiments. Wire 466, wire 468, and wire 470 are circular shaped and are positioned on first body portion 452. Wire 466, wire 468, and wire 470 have different diameters and are concentrically positioned on first body portion 452. Opening 472 extends through a center of first body portion 462 and second body portion 464.

Strain gauge sensor 460 is a second example of a volume displacement sensor that can be used on delivery mechanism 204 (shown in FIG. 11). Opening 472 can be positioned around plunger rod 420 of plunger 406 and strain gauge sensor 460 can be positioned against barrel flange 414 of barrel 402. Strain gauge sensor 460 will thus act as volume displacement sensor 440 and can sense the force placed on it by the compression of spring 442 of delivery mechanism 204. A volume of fluid that has been injected into the inflatable balloon can be determined from the force sensed by strain gauge sensor 460 using Hooke's law.

Strain gauge sensor 450 shown in FIGS. 12A-12B and strain gauge sensor 460 shown in FIGS. 13A-13B are two examples of volume displacement sensors that can be positioned on delivery mechanism 204 to determine a volume of fluid that has been injected into an inflatable balloon of a catheter. In alternate embodiments, other sensors can be used, including force sensors, optical encoders, magnetic encoders, or capacitive encoders.

Strain gauge sensor 450 and strain gauge sensor 460 sandwich strain gauge wires (wire 456 of strain gauge sensor 450 and wire 466, wire 468, and wire 470 of strain gauge sensor 460) between two flexible slabs of compliant material (first body portion 452 and second body portion 454 of strain gauge sensor 450 and first body portion 462 and second body portion 464 of strain gauge sensor 460). Strain gauge sensor 450 and strain gauge sensor 460 are deformed orthogonal to the applied force due to Poisson's effect. The strain sensed by strain gauge sensor 450 and strain gauge sensor 460 can then be used to determine the volume of fluid that has been injected into an inflatable balloon of a catheter using Hooke's law.

Strain gauge sensor 450 and strain gauge sensor 460 can be coupled by a wired or wireless communication link to controller 206 of hyperinflation prevention system 200 (shown in FIG. 2). For example, a wire can extend from wire 456 of strain gauge sensor 450 to controller 206, and a wire can extend from wire 466, wire 468, and wire 470 of strain gauge sensor 460 to controller 206. The strain sensed by strain gauge sensor 450 or strain gauge sensor 460 is communicated to controller 206. The strain sensed by strain gauge sensor 450 or strain gauge sensor 460 is the injected volume signal that is communicated to controller 206. Controller 206 can use the strain sensed by strain gauge sensor 450 or strain gauge sensor 460 to determine the injected volume of fluid in the inflatable balloon based using Hooke's law. Using strain gauge sensor 450 or strain gauge sensor 460 allows delivery mechanism 204 to be used in hyperinflation prevention system 200.

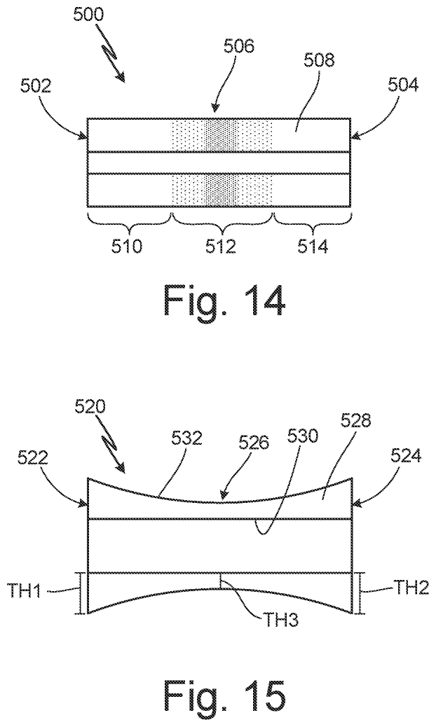

Inflatable balloons 500, 520, 540, 560, and 600 are discussed in FIGS. 14-18B below. Inflatable balloons 500, 520, 540, 560, and 600 are designed to have varying compliance with a higher compliance in a center of inflatable balloons 500, 520, 540, 560, and 600 and a lower compliance at the ends of inflatable balloons 500, 520, 540, 560, and 600. When inflatable balloons 500, 520, 540, 560, and 600 are inflated, the higher compliance center of inflatable balloons 500, 520, 540, 560, and 600 will inflate before the lower compliance ends of inflatable balloons 500, 520, 540, 560, and 600. Inflatable balloons 500, 520, 540, 560, and 600 are further designed to attenuate the pop-open effect of inflatable balloons 500, 520, 540, 560, and 600.

FIG. 14 is a cross-sectional view of inflatable balloon 500. Inflatable balloon 500 includes proximal end 502, distal end 504, center 506, balloon wall 508, proximal section 510, center section 512, and distal section 514.

Inflatable balloon 500 includes proximal end 502 and distal end 504 positioned opposite of proximal end 502. Center 506 of inflatable balloon 500 is positioned between proximal end 502 and distal end 504 of inflatable balloon 500. Inflatable balloon 500 includes balloon wall 508 that extends from proximal end 502 to distal end 504. Balloon wall 508 has a constant thickness from proximal end 502 to distal end 504. Inflatable balloon 500 includes proximal section 510, center section 512, and distal section 514. Proximal section 510 extends from proximal end 502 to center section 512; center section 512 extends from proximal section 510 to distal section 514; and distal section 514 extends from center section 512 to distal end 504.

Inflatable balloon 500 is made out of any material in which the intrinsic properties of the material can be varied to vary the compliance of the material between proximal end 502 and distal end 504 of inflatable balloon 500. Inflatable balloon 500 is designed so that proximal section 510 and distal section 514 have a lower compliance and center section 512 has a higher compliance. The compliance of inflatable balloon 500 is lowest at proximal end 502 and distal end 504. In proximal section 510, the compliance gradually increases from proximal end 502 to center section 512. In distal section 514, the compliant gradually increased from distal end 504 to center section 512. The compliance of inflatable balloon 500 is highest at center 506 of inflatable balloon 500. In center section 512, the compliance gradually increases from proximal section 510 to center 506 and from distal section 514 to center 506.

Inflatable balloon 500 is a heterogenous inflatable balloon that has a higher compliance at center 506 and a lower compliance at proximal end 502 and distal end 504 in inflatable balloon 500. Inflatable balloon 500 is configured to be used with hyperinflation prevention system 200.

FIG. 15 is a cross-sectional view of inflatable balloon 520. Inflatable balloon 520 includes proximal end 522, distal end 524, center 526, balloon wall 528, inner diameter 530, and outer diameter 532. FIG. 15 shows first thickness TH1, second thickness TH2, and third thickness TH3.

Inflatable balloon 520 includes proximal end 522 and distal end 524 positioned opposite of proximal end 522. Center 526 of inflatable balloon 520 is positioned between proximal end 522 and distal end 524 of inflatable balloon 520. Inflatable balloon 520 includes balloon wall 528 that extends from proximal end 522 to distal end 524. Balloon wall 528 has a varying thickness from proximal end 522 to distal end 524. Inflatable balloon 520 has inner diameter 530 and outer diameter 532. Inner diameter 530 is constant from proximal end 522 to distal end 524. Outer diameter 532 varies between proximal end 522 and distal end 524 to vary the thickness of balloon wall 528. As shown in FIG. 15, balloon wall 528 has first thickness TH1 at proximal end 522, second thickness TH2 at distal end 524, and third thickness TH3 at center 526.

Outer diameter 532 is largest at proximal end 522 and distal end 524, thus the thickness of balloon wall 528 is largest at first thickness TH1 and second thickness TH2. Outer diameter 532 is smallest at center 526, thus the thickness of balloon wall 528 is smallest at third thickness TH3. The thickness of balloon wall 528 tapers from the largest thickness at first thickness TH1 at proximal end 522 to the smallest thickness at third thickness TH3 at center 526. The thickness of balloon wall 528 also tapers from the largest thickness at second thickness TH2 at distal end 524 to the smallest thickness at third thickness TH3 at center 526.

The compliance of inflatable balloon 520 varies based on the thickness of balloon wall 528. The compliance of inflatable balloon 520 is highest at center 526 where balloon wall 528 is thinnest. The compliance of inflatable balloon 520 is lowest at proximal end 522 and distal end 524 where balloon wall 528 is thickest. The compliance of inflatable balloon 520 gradually decreases from center 526 to proximal end 522 and from center 526 to distal end 524 as the thickness of balloon wall 528 increases.

Inflatable balloon 520 is a heterogenous inflatable balloon that has a higher compliance at center 526 and a lower compliance at proximal end 522 and distal end 524 in inflatable balloon 520. Inflatable balloon 520 is configured to be used with hyperinflation prevention system 200.

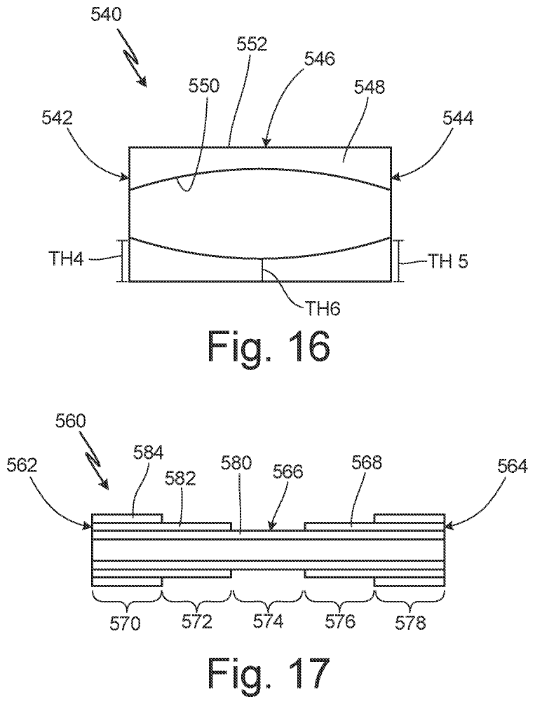

FIG. 16 is a cross-sectional view of inflatable balloon 540. Inflatable balloon 540 includes proximal end 542, distal end 544, center 546, balloon wall 548, inner diameter 550, and outer diameter 552. FIG. 15 shows first thickness TH4, second thickness TH5, and third thickness TH6.

Inflatable balloon 540 includes proximal end 542 and distal end 544 positioned opposite of proximal end 542. Center 546 of inflatable balloon 540 is positioned between proximal end 542 and distal end 544 of inflatable balloon 540. Inflatable balloon 540 includes balloon wall 548 that extends from proximal end 542 to distal end 544. Balloon wall 548 has a varying thickness from proximal end 542 to distal end 544. Inflatable balloon 540 has inner diameter 550 and outer diameter 552. Outer diameter 552 is constant from proximal end 542 to distal end 544. Inner diameter 550 varies between proximal end 542 and distal end 544 to vary the thickness of balloon wall 548. As shown in FIG. 16, balloon wall 548 has first thickness TH4 at proximal end 542, second thickness TH5 at distal end 544, and third thickness TH6 at center 546.

Inner diameter 550 is smallest at proximal end 542 and distal end 544, thus the thickness of balloon wall 548 is largest at first thickness TH4 and second thickness TH5. Inner diameter 550 is largest at center 546, thus the thickness of balloon wall 548 is smallest at third thickness TH6. The thickness of balloon wall 548 tapers from the largest thickness at first thickness TH4 at proximal end 542 to the smallest thickness at third thickness TH6 at center 546. The thickness of balloon wall 548 also tapers from the largest thickness at second thickness TH5 at distal end 544 to the smallest thickness at third thickness TH6 at center 546.

The compliance of inflatable balloon 540 varies based on the thickness of balloon wall 548. The compliance of inflatable balloon 540 is highest at center 546 where balloon wall 548 is thinnest. The compliance of inflatable balloon 540 is lowest at proximal end 542 and distal end 544 where balloon wall 548 is thickest. The compliance of inflatable balloon 540 gradually decreases from center 546 to proximal end 542 and from center 546 to distal end 544 as the thickness of balloon wall 548 increases.

Inflatable balloon 540 is a heterogenous inflatable balloon that has a higher compliance at center 546 and a lower compliance at proximal end 542 and distal end 544 in inflatable balloon 540. Inflatable balloon 540 is configured to be used with hyperinflation prevention system 200.

FIG. 17 is a cross-sectional view of inflatable balloon 560. Inflatable balloon 560 includes proximal end 562, distal end 564, center 566, balloon wall 568, first section 570, second section 572, third section 574, fourth section 576, and fifth section 578. Balloon wall 568 includes first layer 580, second layer 582, and third layer 584.

Inflatable balloon 560 includes proximal end 562 and distal end 564 positioned opposite of proximal end 562. Center 566 of inflatable balloon 560 is positioned between proximal end 562 and distal end 564 of inflatable balloon 560. Inflatable balloon 560 includes balloon wall 568 that extends from proximal end 562 to distal end 564. Balloon wall 568 has a varying thickness from proximal end 562 to distal end 564. Inflatable balloon 560 includes first section 570, second section 572, third section 574, fourth section 576, and fifth section 578. First section 570 extends from proximal end 562 to second section 572; second section extends from first section 570 to third section 574; third section 574 extends from second section 572 to fourth section 576; fourth section 576 extends from third section 574 to fifth section 578; and fifth section 578 extends from fourth section 576 to distal end 564.

Balloon wall 568 includes multiple layers of material. Balloon wall 568 includes first layer 580 that extends from proximal end 562 to distal end 564 across first section 570, second section 572, third section 574, fourth section 576, and fifth section 578. Balloon wall 568 also includes second layer 582 that extends from proximal end 562 across first section 570 and second section 572 and from distal end 564 across fifth section 578 and fourth section 576. Balloon wall 568 further includes third layer 584 that extends from proximal end 562 across first section 570 and from distal end 564 across fifth section 578. First section 570 and fifth section 578 includes three layers of material with first layer 580 forming an inner layer of balloon wall 568, second layer 582 forming a middle layer of balloon wall 568, and third layer 584 forming an outer layer of balloon wall 568. Second section 572 and fourth section 576 include two layers of material with first layer 580 forming an inner layer of balloon wall 568 and second layer 582 forming an outer layer of balloon wall 568. Third section 574 includes a single layer of material with first layer 580 forming the single layer of balloon wall 568. Balloon wall 568 is thickest in first section 570 and fifth section 578, is thinnest in third section 574, and has a medium thickness in second section 572 and fourth section 576.

The compliance of inflatable balloon 560 varies based on the thickness of balloon wall 568. The compliance of inflatable balloon 520 decreases as more balloon wall 568 gets thicker. The compliance of inflatable balloon 520 is highest in third section 574 where balloon wall 568 includes only first layer 580. The compliance of inflatable balloon 520 decreases in second section 572 and fourth section 576 where balloon wall 568 includes first layer 580 and second layer 582. The compliance of inflatable balloon 520 is lowest in first section 570 and fifth section 578 where balloon wall 568 includes first layer 580, second layer 582, and third layer 584.

Inflatable balloon 560 is a heterogenous inflatable balloon that has a higher compliance at center 566 and a lower compliance at proximal end 562 and distal end 564 in inflatable balloon 560. Inflatable balloon 560 is configured to be used with hyperinflation prevention system 200.

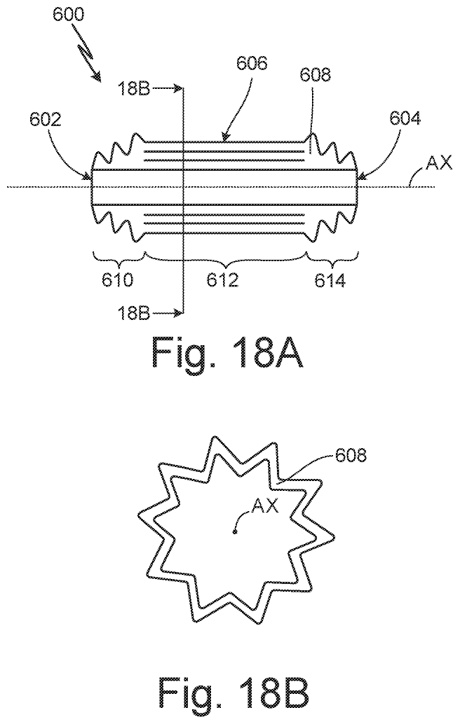

FIG. 18A is a side view of inflatable balloon 600. FIG. 18B is an end view of inflatable balloon 600, taken along line 18B-18B of FIG. 18A. Inflatable balloon 600 includes proximal end 602, distal end 604, center 606, balloon wall 608, proximal section 610, center section 612, and distal section 614. FIGS. 18A-18B also show axis AX.

Inflatable balloon 600 includes proximal end 602 and distal end 604 positioned opposite of proximal end 602. Center 606 of inflatable balloon 600 is positioned between proximal end 602 and distal end 604 of inflatable balloon 600. Inflatable balloon 600 includes balloon wall 608 that extends from proximal end 602 to distal end 604. Inflatable balloon 600 includes proximal section 610, center section 612, and distal section 614. Proximal section 610 extends from proximal end 602 to center section 612; center section 612 extends from proximal section 610 to distal section 614; and distal section 614 extends from center section 612 to distal end 604.

Balloon wall 608 of inflatable balloon 600 is folded. As shown in FIGS. 18A-18B, balloon wall 608 is radially folded in center section 612 around axis AX of inflatable balloon 600. As shown in FIG. 18A, balloon wall 608 is axially folded in proximal section 610 and distal section 614 along axis AX of inflatable balloon 600. As shown in FIG. 18A, balloon wall 608 is folded in proximal section 610 and distal section 614 to form rings that lie on planes normal to axis AX of inflatable balloon 600.

The compliance of inflatable balloon 600 varies based on the folding of balloon wall 608. The folding of inflatable balloon 600 around axis AX in center section 612 has a higher compliance. The folding of inflatable balloon 600 along axis AX in proximal section 610 and distal section 614 has a lower compliance.

Inflatable balloon 600 is a heterogenous inflatable balloon that has a higher compliance at center 606 and a lower compliance at proximal end 602 and distal end 604 in inflatable balloon 600. Inflatable balloon 600 is configured to be used with hyperinflation prevention system 200.

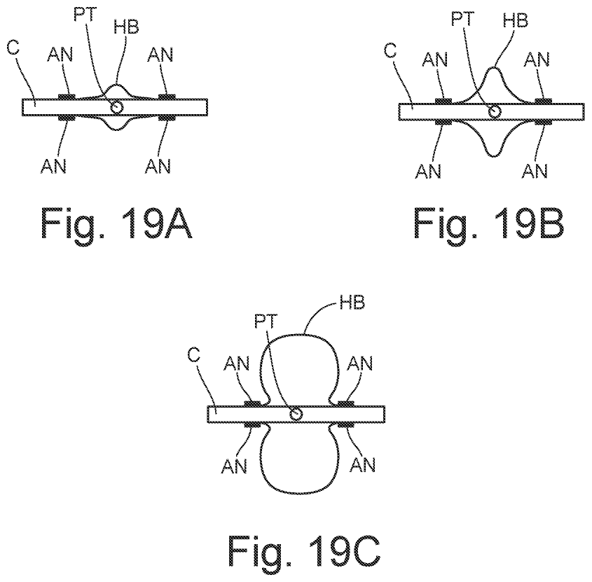

FIG. 19A is a schematic view showing heterogenous inflatable balloon HB on catheter C as it is being inflated. FIG. 19B is a schematic view showing heterogenous inflatable balloon HB on catheter C as a center of heterogenous inflatable balloon HB reaches its maximum stretchability. FIG. 19C is a schematic view showing heterogenous inflatable balloon HB on catheter C as a proximal end and a distal end of heterogenous inflatable balloon HB expand. FIGS. 19A-19C show heterogenous inflatable balloon HB, catheter C, port PT, and anchors AN.

Heterogenous inflatable balloon HB represents any of inflatable balloons 500, 520, 540, 560, and 600 shown in and described in reference to FIGS. 14-18B. Catheter C can be catheter 100 described in FIGS. 1A-1B or any other suitable catheter. Heterogenous inflatable balloon HB and catheter C are shown schematically in FIGS. 19A-19C. Heterogenous inflatable balloon HB is positioned on catheter C. Port PT is an opening between a lumen in catheter C and an interior of heterogeneous balloon HB. Fluid flowing through the lumen in catheter C can enter heterogenous balloon HB through port PT when heterogenous balloon HB is being inflated. Fluid can exit heterogenous balloon HB through port PT when heterogenous balloon HB is being deflated. Heterogeneous balloon HB is anchored to catheter C with anchors AN.

Heterogenous inflatable balloon HB is an inflatable balloon that has a higher compliance in the center and lower compliance at a proximal end and at a distal end, for example any of inflatable balloons 500, 520, 540, 560, and 600. As shown in FIG. 19A, as heterogenous inflatable balloon HB is inflated, the higher compliance center expands first. During this time, the proximal end and the distal end of heterogenous inflatable balloon HB only expand a small amount, if at all. As shown in FIG. 19B, as the pressure in heterogenous inflatable balloon HB increases, the high compliance center reaches its maximum stretchability (which is a lower compliance zone). As shown in FIG. 19C, after the center reaches its maximum stretchability, the lower compliance proximal end and the lower compliance distal end of heterogenous inflatable balloon HB start to stretch until they also reach their maximum stretchability.

Prior art inflatable balloons are homogenous balloons that have a constant compliance from a proximal end to a distal end of the balloon. Further, prior art inflatable balloons have a lower compliance and are typically made out of a stretchable layer of material. As a result, more pressure is needed to expand the inflatable balloon. As the inflatable balloon is inflated and expands, it may over-expand and also cause the blood vessel in which the inflatable balloon is positioned to over-expand. The over-expansion of the blood vessel may cause the blood vessel to aneurysm or rupture. Prior art systems for preventing hyperinflation of inflatable catheter balloons only take into account the pressure inside of the balloon and disregard the pressure the balloon exerts on the vessel wall. This is further complicated due to the low compliance of prior art inflatable catheter balloons.

Heterogenous inflatable balloons HB (which have a varying compliance between the proximal end and the distal end of the inflatable balloon, including any of inflatable balloons 500, 520, 540, 560, or 600) have a higher overall compliance and an attenuated pop-open effect in comparison with prior art homogenous inflatable balloons. Heterogenous inflatable balloon HB requires a lower pressure to expand, as the higher compliance center of heterogenous inflatable balloon HB will expand at lower pressures. The higher compliance of heterogenous inflatable balloons HB also makes it easier to control inflatable balloon hyperinflation.

When heterogenous inflatable balloons HB contact the blood vessel, they are less likely to over-expand and cause blood vessel rupture. This is due to the fact that when heterogenous inflatable balloons HB contact the blood vessel they will have a higher overall compliance, a larger contact area, and a lower pressure in the inflatable balloon. A larger contact area ensures better wedging of the blood vessel and better sensing of the mechanical properties of the blood vessel. Further, heterogenous inflatable balloons HB will be more sensitive to the effect of blood vessel loading and therefore to the estimation of the mechanical properties of the blood vessel. Knowing the mechanical properties of the blood vessel can help guide treatment decisions. For example, measuring and monitoring the elasticity of blood vessels can have diagnostic value when administering vasoactive drugs to treat pulmonary hypertension.

Further, the shape formed when heterogenous inflatable balloons HB are inflated offer superior resistance to the blood flow in comparison to convention homogenous inflatable balloons. This will help float heterogenous inflatable balloons HB from the right atrium through the right ventricle and into the pulmonary arteries.

As shown above in FIGS. 3A-4, prior art inflatable balloons have a pop-open effect at a certain pressure in which the diameter of the inflatable balloon quickly rises and the pressure in the inflatable balloon drops. Due to the sudden drop in pressure, it is difficult for a physician to sense the pressure inside the balloon and to control the safe amount of injected volume of fluid in the inflatable balloon. Heterogenous inflatable balloons HB are designed to have a negligible pop-open effect. Further, heterogenous inflatable balloons HB with higher compliance require a lower pressure to inflate and are less likely to damage the blood vessel if the catheter is accidently pulled while heterogenous inflatable balloon HB is still inflated.

Prior art inflatable balloons were designed with lower compliance so the inflatable balloon could drive the retraction of the injection device (for example, a plunger of a syringe) that is used to inflate the inflatable balloon. Heterogenous inflatable balloons HB with a higher overall compliance can be safely used in combination with hyperinflation prevention system 200. Delivery mechanism 204, shown in and discussed in reference to FIGS. 2 and 11 above, has a spring-retractable plunger that drives the retraction of the injection device in combination with valve mechanism 202, shown in and discussed in reference to FIGS. 2 and 7-10B above. As such, hyperinflation prevention system 200 does not rely on the inflatable balloon to drive retraction of delivery mechanism 204.

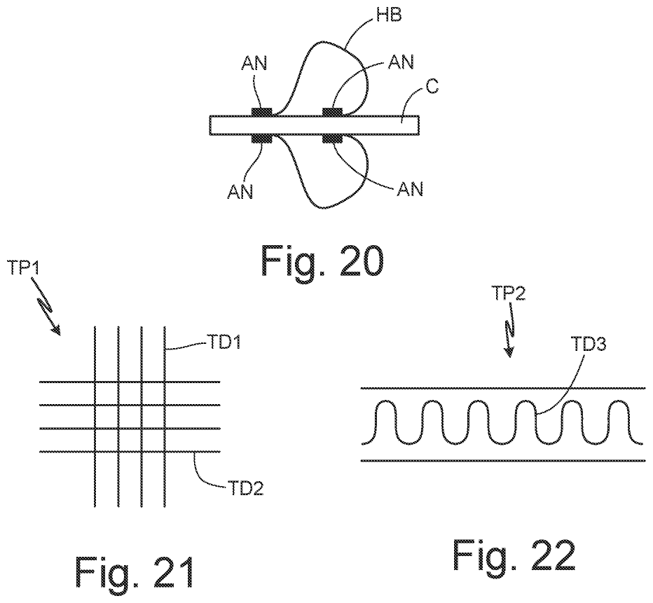

FIG. 20 is a schematic view showing heterogenous inflatable balloon HB anchored to catheter C. FIG. 20 shows heterogenous inflatable balloon HB, anchors AN, and catheter C.

Heterogenous inflatable balloon HB represents any of inflatable balloons 500, 520, 540, 560, and 600 shown in and described in reference to FIGS. 14-18B. Inflatable balloons are typically anchored to a catheter on an exterior of the inflatable balloon, as shown in FIGS. 19A-19C. As shown in FIG. 20, heterogenous inflatable balloon HB can also be anchored to catheter C on an interior of heterogenous inflatable balloon HB. A proximal end of heterogenous inflatable balloon HB is anchored to catheter C with anchors AN on an exterior of heterogenous inflatable balloon HB, and a distal end of heterogenous inflatable balloon HB is anchored to catheter C with anchors AN on an interior of heterogenous inflatable balloon HB. In an alternate embodiment, the proximal end or both the proximal end and the distal end of heterogenous inflatable balloon HB can be anchored to catheter C on an interior of heterogenous inflatable balloon HB.

Anchoring heterogenous inflatable balloon HB to catheter C on an interior of heterogeneous inflatable balloon HB changes the inflation pattern of heterogenous inflatable balloon HB. Heterogenous inflatable balloon HB can be anchored to catheter C on an interior of heterogenous inflatable balloon HB to modify the inflation pattern of heterogenous balloon HB so it wedges more easily in a patient's pulmonary artery.

FIG. 21 is a schematic top view of thread pattern TP1 for a heterogenous inflatable balloon. FIG. 22 is a schematic cross-sectional view of thread pattern TP2 for a heterogenous inflatable balloon. FIG. 21 shows thread pattern TP1, which includes first threads TD1 and second threads TD2. FIG. 22 shows thread pattern TP2, which includes thread TD3.

Thread pattern TP1 and thread pattern TP2 are thread patterns that can be used for any inflatable balloon, including any of heterogeneous inflatable balloons 500, 520, 540, 560, and 600. FIG. 21 shows a schematic top view of thread pattern TP1. Thread pattern TP1 includes first threads TD1 running in a first direction and second threads TD2 running in a second direction. First threads TD1 and second threads TD2 are in a crisscross pattern. FIG. 22 shows a schematic cross-sectional view of thread pattern TP2. Thread pattern TP2 includes thread TD3 in a serpentine pattern.

To mitigate the changes of rupture of a heterogenous inflatable balloon having a higher compliance (including inflatable balloons 500, 520, 540, 560, and 600), the heterogenous inflatable balloon can be reinforced with threads in thread pattern TP1 or threads in thread pattern TP2. Thread pattern TP1 and thread pattern TP2 improve the strength of the heterogeneous inflatable balloon.

The following are non-exclusive descriptions of possible embodiments of the present invention.

A system for preventing hyperinflation of an inflatable balloon of a catheter includes an injection device that is configured to be fluidly coupled to the catheter to inject a fluid into the inflatable balloon of the catheter, and a sensing device that is configured to sense an injected volume of the fluid in the inflatable balloon of the catheter. A controller is coupled by a wired or wireless communication link to the sensing device to receive an injected volume signal from the sensing device that indicates the injected volume of the fluid in the inflatable balloon of the catheter. The controller is configured to determine an actuation pressure based on the injected volume of the fluid in the inflatable balloon. A pressure relief valve is fluidly coupled to the catheter and coupled by a wired or wireless communication link to the controller. The controller is configured to adjust the actuation pressure of the pressure relief valve based on the injected volume of the fluid in the inflatable balloon.

The system of the preceding paragraph can optionally include, additionally and/or alternatively, any one or more of the following features, configurations and/or additional components:

Wherein the controller is configured to determine the actuation pressure based on the injected volume of the fluid in the inflatable balloon using a threshold pressure versus injected volume curve.

Wherein the actuation pressure on the threshold pressure versus injected volume curve is dependent upon the injected volume of the fluid in the inflatable balloon.

Wherein the threshold pressure versus injected volume curve is set based on a pressure versus injected volume curve of an unloaded inflatable balloon.

Wherein the threshold pressure versus injected volume curve is positively offset from the pressure versus injected volume curve of the unloaded inflatable balloon.

Wherein the threshold pressure versus injected volume curve allows for higher pressures for a given injected volume when compared to the pressure versus injected volume curve of the unloaded inflatable balloon.

Wherein the controller is configured to use the injected volume signal to determine the injected volume of fluid in the inflatable balloon.

Wherein the actuation pressure of the pressure relief valve is a pressure at which the pressure relief valve will be actuated.

The system further includes a pressure sensor fluidly coupled to the catheter that is configured to sense a pressure in the inflatable balloon of the catheter, wherein the controller is coupled by a wired or wireless communication link to the pressure sensor to receive a pressure signal from the pressure sensor.

Wherein the controller is configured to use the pressure signal to determine the pressure of the fluid in the inflatable balloon and whether the pressure of the fluid in the inflatable balloon exceeds the actuation pressure.

Wherein the injection device is coupled by a wired or wireless communication link to the controller, and wherein the controller is configured to actuate the injection device when the actuation pressure has been exceeded.

Wherein the pressure relief valve and the pressure sensor are an integral unit.

Wherein the injection device and the sensing device are an integral unit.

The system further includes a check valve fluidly coupled to the catheter that is configured to prevent a negative pressure in the catheter.

Wherein the check valve and the pressure relief valve are an integral unit.

Wherein the sensing device is electrically, mechanically, or fluidly connected to the injection device.

Wherein the pressure relief valve is a valve mechanism that includes a disposable part and a reusable part that is configured to releasably connect to the disposable part. The disposable part includes a body having a cavity, a first end, and a second end opposite the first end, a tube extending through the first end of the body and into the cavity in the body, and a first disk seal sealed in an opening at the second end of the body. A first end of the tube is configured to be fluidly coupled to the catheter. The reusable part includes a body having a cavity, a first end, and a second end opposite the first end, a second disk seal sealed in an opening at the first end of the body, a linear actuator in the cavity of the body, and a spring extending between the linear actuator and the second disk seal.

Wherein when the valve mechanism is in a closed position, the first disk seal is deformed and sealed against a second end of the tube in the disposable part, and wherein when the valve mechanism is in an open position, a second end of the tube is fluidly coupled to the cavity of the body of the disposable part.

Wherein the injection device further comprises a syringe that is configured to inject fluid into the inflatable balloon of the catheter, wherein the syringe includes a barrel and a plunger positioned in the barrel, and a locking mechanism that is configured to lock the plunger of the syringe in position in the barrel of the syringe.

The system further includes the catheter having the inflatable balloon, wherein the inflatable balloon has a varying compliance from a proximal end to a distal end of the inflatable balloon.

A method of preventing hyperinflation of an inflatable balloon of a catheter includes injecting a fluid into the inflatable balloon of the catheter with an injection device and measuring an injected volume of the fluid in the inflatable balloon of the catheter with a sensing device. An injected volume signal is sent from the sensing device to a controller, wherein the injected volume signal indicates the injected volume of the fluid in the inflatable balloon. The controller determines an actuation pressure based on the injected volume of the fluid in the inflatable balloon. The actuation pressure of a pressure relief valve is adjusted based on the injected volume of the fluid in the inflatable balloon.

The method of the preceding paragraph can optionally include, additionally and/or alternatively, any one or more of the following features, configurations and/or additional components:

Wherein determining, in the controller, the actuation pressure based on the injected volume of the fluid in the inflatable balloon includes determining the actuation pressure based on a threshold pressure versus injected volume curve.

Wherein the actuation pressure on the threshold pressure versus injected volume curve is dependent upon the injected volume of the fluid in the inflatable balloon of the catheter.

The method further includes measuring a pressure versus injected volume curve of an unloaded inflatable balloon, and setting the threshold pressure versus injected volume curve based on the pressure versus injected volume curve of the unloaded inflatable balloon.

Wherein the threshold pressure versus injected volume curve is positively offset from the pressure versus injected volume curve of the unloaded inflatable balloon.

The method further includes sensing a pressure in the inflatable balloon of the catheter with a pressure sensor; sending a pressure signal from the pressure sensor to the controller, wherein the pressure signal indicates the pressure in the inflatable balloon of the catheter; and determining, in the controller, whether the pressure in the inflatable balloon of the catheter exceeds the actuation pressure.

The method further includes actuating the injection device to withdraw fluid from the inflatable balloon of the catheter.

The method further includes signaling an alarm when the pressure in the inflatable balloon of the catheter exceeds the actuation pressure.

The method further includes sensing a spike in pressure in the inflatable balloon of the catheter that exceeds the actuation pressure when the inflatable balloon of the catheter is pulled while inflated, and actuating the pressure relief valve and/or the injection device to reduce the pressure and/or the injected volume of the fluid in the inflatable balloon of the catheter.

A system for preventing hyperinflation of an inflatable balloon of a catheter includes an injection device that is configured to be fluidly coupled to the catheter to inject a fluid into the inflatable balloon of the catheter, and a sensing device that is configured to sense an injected volume of the fluid in the inflatable balloon of the catheter. A controller is coupled by a wired or wireless communication link to the sensing device to receive an injected volume signal from the sensing device that indicates the injected volume of the fluid in the inflatable balloon of the catheter. The controller is configured to determine an actuation pressure based on the injected volume of the fluid in the inflatable balloon of the catheter.

The system of the preceding paragraph can optionally include, additionally and/or alternatively, any one or more of the following features, configurations and/or additional components:

Wherein the injection device further includes a syringe that is configured to inject fluid into the inflatable balloon of the catheter, wherein the syringe includes a barrel and a plunger positioned in the barrel, and a locking mechanism that is configured to lock the plunger of the syringe in position in the barrel of the syringe.

Wherein the plunger of the syringe includes teeth on a rod of the plunger.

Wherein the locking mechanism further includes a body positioned in the barrel of the syringe, teeth positioned on the body of the locking mechanism, and a trigger connected to the body of the locking mechanism in the barrel of the syringe and extending outside of the barrel of the syringe.

Wherein the teeth on the body of the locking mechanism are configured to engage the teeth on the rod of the plunger to lock the plunger in position in the barrel.

Wherein the trigger is configured to move the body and the teeth of the locking mechanism inwards to engage the teeth on the rod of the plunger.

Wherein the trigger is positioned under a barrel flange of the barrel of the syringe.

Wherein the locking mechanism further includes a spring extending between the trigger of the locking mechanism and the barrel flange of the barrel of the syringe.

Wherein when the trigger is pressed to overcome a spring bias of the spring, the locking mechanism is configured to be released.

The system further includes a spring positioned around a rod of the plunger outside of the barrel, wherein the sensing device is a volume displacement sensor positioned between a barrel flange of the barrel and the spring on the rod of the plunger.

Wherein the volume displacement sensor is configured to sense the injected volume of fluid in the inflatable balloon of the catheter.

Wherein the volume displacement sensor is a strain gauge sensor that is positioned around the rod of the plunger.

Wherein the strain gauge sensor is configured to sense a strain being placed on the strain gauge sensor based on a load of the spring.

Wherein the strain gauge sensor is configured to communicate the strain sensed in the strain gauge sensor to the controller, and wherein the controller is configured to determine the injected volume of fluid in the inflatable balloon of the catheter based on the strain sensed in the strain gauge sensor.

Wherein the strain gauge sensor further includes a first body portion of compliant material, a second body portion of compliant material, and a wire positioned between the first body portion and the second body portion.

Wherein the wire has a wavy shape and is positioned in a circle between the first body portion and the second body portion.

Wherein the strain gauge sensor includes a plurality of wires positioned in concentric circles.

Wherein the syringe further includes a displacement limiter positioned on an interior surface of the barrel, wherein the displacement limiter is configured to prevent the plunger from being displaced beyond a predetermined distance.

Wherein the injection device and the sensing device are an integral unit.

The system further includes a pressure relief valve fluidly coupled to the catheter and coupled by a wired or wireless communication link to the controller, wherein the controller is configured to adjust the actuation pressure of the pressure relief valve based on the injected volume of the fluid in the inflatable balloon.

Wherein the controller is configured to determine the actuation pressure based on the injected volume of the fluid in the inflatable balloon using a threshold pressure versus injected volume curve.

Wherein the actuation pressure on the threshold pressure versus injected volume curve is dependent upon the injected volume of the fluid in the inflatable balloon.

Wherein the threshold pressure versus injected volume curve is set based on a pressure versus injected volume curve of an unloaded inflatable balloon.

Wherein the threshold pressure versus injected volume curve is positively offset from the pressure versus injected volume curve of the unloaded inflatable balloon.

A catheter system includes a catheter having an inflatable balloon, an injection device fluidly coupled to the catheter that is configured to inject a fluid into the inflatable balloon of the catheter, and a sensing device that is configured to sense an injected volume of the fluid in the inflatable balloon of the catheter. A controller is coupled by a wired or wireless communication link to the sensing device to receive an injected volume signal from the sensing device that indicates the injected volume of the fluid in the inflatable balloon of the catheter. The controller is configured to determine an actuation pressure based on the injected volume of the fluid in the inflatable balloon. A pressure relief valve is fluidly coupled to the catheter and coupled by a wired or wireless communication link to the controller. The controller is configured to adjust the actuation pressure of the pressure relief valve based on the injected volume of the fluid in the inflatable balloon.

The catheter system of the preceding paragraph can optionally include, additionally and/or alternatively, any one or more of the following features, configurations and/or additional components:

Wherein the inflatable balloon has a varying compliance from a proximal end to a distal end of the inflatable balloon.

Wherein the inflatable balloon has a lower compliance at the proximal end and the distal end of the inflatable balloon and a higher compliance at a center of the inflatable balloon.

Wherein the inflatable balloon has a varying compliance from the proximal end to the distal end of the inflatable balloons based on intrinsic properties of the inflatable balloon.

Wherein the inflatable balloon has a constant thickness from the proximal end to the distal end of the inflatable balloon.

Wherein the inflatable balloon has a varying thickness from the proximal end to the distal end of the inflatable balloon.

Wherein a wall of the inflatable balloon is thickest at the proximal end and the distal end of the inflatable balloon and thinnest at the center of the inflatable balloon.

Wherein an inner diameter of the inflatable balloon is constant from the proximal end to the distal end of the inflatable balloon, and wherein an outer diameter of the inflatable balloon varies from the proximal end to the distal end of the inflatable balloon.

Wherein an outer diameter of the inflatable balloon is constant from the proximal end to the distal end of the inflatable balloon, and wherein an inner diameter of the inflatable balloon varies from the proximal end to the distal end of the inflatable balloon.

Wherein the inflatable balloon includes multiple layers.

Wherein the inflatable balloon includes three layers in a first section adjacent the proximal end and a fifth section adjacent the distal end, two layers in a second section adjacent the first section and a fourth section adjacent the fifth section, and one layer in a third section adjacent the second section and the fourth section.

Wherein the compliance of the inflatable balloon is lowest in the first section and the fifth section and highest in the third section.

Wherein a proximal section near the proximal end of the inflatable balloon and a distal section near the distal end of the inflatable balloon have a lower compliance than a center section of the inflatable balloon.

Wherein a wall of the inflatable balloon is axially folded in the proximal section and the distal section, and wherein the wall of the inflatable balloon is radially folded in the center section.

Wherein the proximal end, the distal end, or both the proximal end and the distal end of the inflatable balloon are anchored to the catheter on an interior of the inflatable balloon.

Wherein the inflatable balloon has threads in a crisscross pattern.

Wherein the inflatable balloon has threads in a serpentine pattern.

Wherein the controller is configured to determine the actuation pressure based on the injected volume of the fluid in the inflatable balloon using a threshold pressure versus injected volume curve.

Wherein the actuation pressure on the threshold pressure versus injected volume curve is dependent upon the injected volume of the fluid in the inflatable balloon.

Wherein the threshold pressure versus injected volume curve is set based on a pressure versus injected volume curve of an unloaded inflatable balloon.

Wherein the threshold pressure versus injected volume curve is positively offset from the pressure versus injected volume curve of the unloaded inflatable balloon.

Wherein the actuation pressure of the pressure relief valve is a pressure at which the pressure relief valve will be actuated.

A system for preventing hyperinflation of an inflatable balloon of a catheter includes a pressure relief valve fluidly coupled to the inflatable balloon and the catheter, and a controller coupled by a wired or wireless communication link to the pressure relief valve. The controller is configured to send a signal to the pressure relief valve to dynamically adjust an actuation pressure of the pressure relief valve. The actuation pressure is dependent on an injected volume of fluid in the inflatable balloon of the catheter.

The system of the preceding paragraph can optionally include, additionally and/or alternatively, any one or more of the following features, configurations and/or additional components:

Wherein the controller is configured to determine the actuation pressure based on the injected volume of the fluid in the inflatable balloon using a threshold pressure versus injected volume curve.

Wherein the actuation pressure on the threshold pressure versus injected volume curve is dependent upon the injected volume of the fluid in the inflatable balloon.

Wherein the threshold pressure versus injected volume curve is set based on a pressure versus injected volume curve of an unloaded inflatable balloon.

Wherein the threshold pressure versus injected volume curve is positively offset from the pressure versus injected volume curve of the unloaded inflatable balloon.

Wherein the actuation pressure of the pressure relief valve is a pressure at which the pressure relief valve will be actuated.

Wherein the pressure relief valve includes a disposable part that is releasably connectable to a reusable part.

Wherein the pressure relief valve is a valve mechanism that includes a disposable part and a reusable part that is configured to releasably connect to the disposable part. The disposable part includes a body having a cavity, a first end, and a second end opposite the first end, a tube extending through the first end of the body and into the cavity in the body, and a first disk seal sealed in an opening at the second end of the body. A first end of the tube is configured to be fluidly coupled to the catheter. The reusable part includes a body having a cavity, a first end, and a second end opposite the first end, a second disk seal sealed in an opening at the first end of the body, a linear actuator in the cavity of the body, and a spring extending between the linear actuator and the second disk seal.

Wherein when the valve mechanism is in a closed position, the first disk seal is deformed and sealed against a second end of the tube in the disposable part.

Wherein when the valve mechanism is in an open position, a second end of the tube is fluidly coupled to the cavity of the body of the disposable part.

Wherein the disposable part further includes an outlet in the body of the disposable part, wherein when the valve mechanism is in an open position, the second end of the tube is fluidly coupled to the cavity in the body of the disposable part and the outlet in the body of the disposable part.

Wherein when the disposable part is connected to the reusable part, the first disk seal and the second disk seal are coupled together with a coupling between the first disk seal and the second disk seal.

Wherein the coupling between the first disk seal and the second disk seal is a magnetic coupling.

Wherein the coupling between the first disk seal and the second disk seal is a mechanical coupling.

Wherein the linear actuator is configured to place a load on the spring to place a force on the second disk seal to deform the second disk seal and the first disk seal.

Wherein the load placed on the spring by the linear actuator determines the actuation pressure needed to actuate the valve mechanism.

Wherein the valve mechanism is configured to be actuated when a pressure in the catheter exceeds the actuation pressure of the valve mechanism.

Wherein the linear actuator is coupled by a wired or wireless communication link to the controller.

Wherein the controller is configured to send a signal to the linear actuator to vary the load placed on the spring to vary the actuation pressure of the valve mechanism.

Wherein the valve mechanism further includes a pressure sensor positioned in the tube.

Wherein the pressure sensor is coupled by a wired or wireless communication link to a controller and is configured to sense a pressure in the catheter.

The system further includes an injection device that is configured to be fluidly coupled to the catheter to inject the fluid into the inflatable balloon of the catheter, and a sensing device that is configured to sense the injected volume of the fluid in the inflatable balloon of the catheter, wherein the sensing device is coupled by a wired or wireless communication link to the controller and is configured to send an injected volume signal to the controller.

A method of preventing hyperinflation of an inflatable balloon of a catheter includes determining, in a controller, an actuation pressure based on an injected volume of a fluid in the inflatable balloon of the catheter. A signal is sent from the controller to a pressure relief valve. The actuation pressure of the pressure relief valve is adjusted based on the signal from the controller, wherein the controller is configured to dynamically adjust the actuation pressure of the pressure relief valve.

The method of the preceding paragraph can optionally include, additionally and/or alternatively, any one or more of the following features, configurations and/or additional components:

Wherein determining, in the controller, the actuation pressure based on the injected volume of the fluid in the inflatable balloon includes determining the actuation pressure based on a threshold pressure versus injected volume curve.

Wherein the actuation pressure on the threshold pressure versus injected volume curve is dependent upon the injected volume of the fluid in the inflatable balloon of the catheter.

The method further includes measuring a pressure versus injected volume curve of an unloaded inflatable balloon, and setting the threshold pressure versus injected volume curve based on the pressure versus injected volume curve of the unloaded inflatable balloon.

Wherein sending the signal from the controller to the pressure relief valve includes sending the signal from the controller to a linear actuator of the pressure relief valve.

Wherein adjusting the actuation pressure of the pressure relief valve includes adjusting a load being placed on a spring with a linear actuator of the pressure relief valve.

The method further includes actuating the pressure relief valve when a pressure in the catheter exceeds the actuation pressure of the pressure relief valve.

Wherein actuating the pressure relief valve includes overcoming a load of a spring to move a first disk seal to an undeformed state.

The method further includes injecting the fluid into the inflatable balloon of the catheter with an injection device, measuring the injected volume of the fluid in the inflatable balloon of the catheter with a sensing device, and sending an injected volume signal from the sensing device to the controller, wherein the injected volume signal indicates the injected volume of the fluid in the inflatable balloon.

A valve mechanism includes a disposable part and a reusable part that is configured to releasably connect to the disposable part. The disposable part includes a body having a cavity, a first end, and a second end opposite the first end, a tube extending through the first end of the body and into the cavity in the body, and a first disk seal sealed in an opening at the second end of the body. A first end of the tube is configured to be fluidly coupled to a catheter. The reusable part includes a body having a cavity, a first end, and a second end opposite the first end, a second disk seal sealed in an opening at the first end of the body, a linear actuator in the cavity of the body, and a spring extending between the linear actuator and the second disk seal. The linear actuator is configured to adjust a load placed on the spring to adjust an actuation pressure of the valve mechanism.

The valve mechanism of the preceding paragraph can optionally include, additionally and/or alternatively, any one or more of the following features, configurations and/or additional components:

Wherein when the valve mechanism is in a closed position, the first disk seal is deformed and sealed against a second end of the tube in the disposable part.

Wherein when the valve mechanism is in an open position, a second end of the tube is fluidly coupled to the cavity of the body of the disposable part.

Wherein the disposable part further includes an outlet in the body of the disposable part, wherein when the valve mechanism is in an open position, the second end of the tube is fluidly coupled to the cavity in the body of the disposable part and the outlet in the body of the disposable part.

Wherein when the disposable part is connected to the reusable part, the first disk seal and the second disk seal are coupled together with a coupling between the first disk seal and the second disk seal.

Wherein the coupling between the first disk seal and the second disk seal is a magnetic coupling.

Wherein the magnetic coupling includes a ferromagnetic contact on the first disk seal and a magnet on the second disk seal.

Wherein the coupling between the first disk seal and the second disk seal is a mechanical coupling.

Wherein the mechanical coupling includes a first mechanical coupling on the first disk seal and a second mechanical coupling on the second disk seal.

Wherein the load placed on the spring by the linear actuator places a force on the second disk seal to deform the second disk seal and the first disk seal.

Wherein the valve mechanism is configured to be actuated when a pressure in the catheter exceeds the actuation pressure of the valve mechanism.

Wherein the linear actuator is coupled by a wired or wireless communication link to a controller.

Wherein the controller is configured to send a signal to the linear actuator to vary the load placed on the spring to vary the actuation pressure of the valve mechanism.

Wherein the controller is configured to send a signal to the linear actuator to open the valve mechanism if a negative pressure exists in the catheter.

Wherein the reusable part further includes a force sensor between the linear actuator and the spring.

Wherein the force sensor is coupled by a wired or wireless communication link to a controller and is configured to sense the load being placed on the spring by the linear actuator.

Wherein the disposable part further includes a pressure sensor positioned in the tube.

Wherein the pressure sensor is coupled by a wired or wireless communication link to a controller and is configured to sense a pressure in the catheter.

Wherein the disposable part has first threads on the outside of the body of the disposable part and the reusable part has second threads on the inside of the body of the reusable part, and wherein the disposable part and the reusable part are releasably connected with the first threads and the second threads.

A system for preventing hyperinflation of an inflatable balloon of a catheter includes an injection device that is configured to be fluidly coupled to the catheter to inject a fluid into the inflatable balloon of the catheter, and a sensing device that is configured to sense an injected volume of the fluid in the inflatable balloon of the catheter. A controller is coupled by a wired or wireless communication link to the sensing device to receive an injected volume signal from the sensing device that indicates the injected volume of the fluid in the inflatable balloon of the catheter. The controller is configured to determine an actuation pressure based on the injected volume of the fluid in the inflatable balloon of the catheter. A valve mechanism includes a disposable part and a reusable part that is configured to releasably connect to the disposable part. The disposable part includes a body having a cavity, a first end, and a second end opposite the first end, a tube extending through the first end of the body and into the cavity in the body, and a first disk seal sealed in an opening at the second end of the body. A first end of the tube is configured to be fluidly coupled to a catheter. The reusable part includes a body having a cavity, a first end, and a second end opposite the first end, a second disk seal sealed in an opening at the first end of the body, a linear actuator in the cavity of the body, and a spring extending between the linear actuator and the second disk seal. The linear actuator is configured to adjust a load placed on the spring to adjust an actuation pressure of the valve mechanism. The controller is configured to send a signal to the linear actuator to adjust the load placed on the spring to adjust the actuation pressure of the valve mechanism.

The system of the preceding paragraph can optionally include, additionally and/or alternatively, any one or more of the following features, configurations and/or additional components:

Wherein the controller is configured to determine the actuation pressure based on the injected volume of the fluid in the inflatable balloon using a threshold pressure versus injected volume curve.

Wherein the actuation pressure on the threshold pressure versus injected volume curve is dependent upon the injected volume of the fluid in the inflatable balloon.

Wherein the threshold pressure versus injected volume curve is set based on a pressure versus injected volume curve of an unloaded inflatable balloon.

Wherein the threshold pressure versus injected volume curve is positively offset from the pressure versus injected volume curve of the unloaded inflatable balloon.

Wherein when the valve mechanism is in a closed position, the first disk seal is deformed and sealed against a second end of the tube in the disposable part.

Wherein when the valve mechanism is in an open position, a second end of the tube is fluidly coupled to the cavity of the body of the disposable part.

Wherein the disposable part further includes an outlet in the body of the disposable part, wherein when the valve mechanism is in an open position, the second end of the tube is fluidly coupled to the cavity in the body of the disposable part and the outlet in the body of the disposable part.

Wherein when the disposable part is connected to the reusable part, the first disk seal and the second disk seal are coupled together with a coupling between the first disk seal and the second disk seal.

Wherein the coupling between the first disk seal and the second disk seal is a magnetic coupling.

Wherein the magnetic coupling includes a ferromagnetic contact on the first disk seal and a magnet on the second disk seal.

Wherein the coupling between the first disk seal and the second disk seal is a mechanical coupling.

Wherein the mechanical coupling includes a first mechanical coupling on the first disk seal and a second mechanical coupling on the second disk seal.

Wherein the load placed on the spring by the linear actuator places a force on the second disk seal to deform the second disk seal and the first disk seal.

Wherein the valve mechanism is configured to be actuated when a pressure in the catheter exceeds the actuation pressure of the valve mechanism.

Wherein the linear actuator is coupled by a wired or wireless communication link to a controller.

Wherein the controller is configured to send a signal to the linear actuator to vary the load placed on the spring to vary the actuation pressure of the valve mechanism.

Wherein the controller is configured to send a signal to the linear actuator to open the valve mechanism if a negative pressure exists in the catheter.

Wherein the reusable part further includes a force sensor between the linear actuator and the spring.

Wherein the force sensor is coupled by a wired or wireless communication link to a controller and is configured to sense the load being placed on the spring by the linear actuator.

Wherein the disposable part further includes a pressure sensor positioned in the tube.

Wherein the pressure sensor is coupled by a wired or wireless communication link to a controller and is configured to sense a pressure in the catheter.

Wherein the disposable part has first threads on the outside of the body of the disposable part and the reusable part has second threads on the inside of the body of the reusable part, and wherein the disposable part and the reusable part are releasably connected with the first threads and the second threads.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for preventing hyperinflation of an inflatable balloon of a catheter, the system comprising:
an injection device that is configured to be fluidly coupled to the catheter to inject a fluid into the inflatable balloon of the catheter;
a sensing device that is configured to sense an injected volume of the fluid in the inflatable balloon of the catheter;
a controller coupled by a wired or wireless communication link to the sensing device to receive an injected volume signal from the sensing device that indicates the injected volume of the fluid in the inflatable balloon of the catheter; and
a pressure relief valve fluidly coupled to the catheter and coupled by a wired or wireless communication link to the controller, wherein the controller is configured to determine an actuation pressure of the pressure relief valve based on the injected volume of the fluid in the inflatable balloon, and wherein the controller is configured to send a signal to the pressure relief valve to adjust the actuation pressure of the pressure relief valve.

2. The system of claim 1, wherein the controller is configured to determine the actuation pressure based on the injected volume of the fluid in the inflatable balloon using a threshold pressure versus injected volume curve.

3. The system of claim 2, wherein the actuation pressure on the threshold pressure versus injected volume curve is dependent upon the injected volume of the fluid in the inflatable balloon.

4. The system of claim 2, wherein the threshold pressure versus injected volume curve is set based on a pressure versus injected volume curve of an unloaded inflatable balloon.

5. The system of claim 4, wherein the threshold pressure versus injected volume curve is positively offset from the pressure versus injected volume curve of the unloaded inflatable balloon.

6. The system of claim 4, wherein the threshold pressure versus injected volume curve allows for higher pressures for a given injected volume when compared to the pressure versus injected volume curve of the unloaded inflatable balloon.

7. The system of claim 1, wherein the controller is configured to use the injected volume signal to determine the injected volume of fluid in the inflatable balloon.

8. The system of claim 1, wherein the actuation pressure of the pressure relief valve is a pressure at which the pressure relief valve will be actuated.

9. The system of claim 1, and further comprising a pressure sensor fluidly coupled to the catheter that is configured to sense a pressure in the inflatable balloon of the catheter, wherein the controller is coupled by a wired or wireless communication link to the pressure sensor to receive a pressure signal from the pressure sensor.

10. The system of claim 9, wherein the controller is configured to use the pressure signal to determine the pressure of the fluid in the inflatable balloon and whether the pressure of the fluid in the inflatable balloon exceeds the actuation pressure.

11. The system of claim 10, wherein the injection device is coupled by a wired or wireless communication link to the controller, and wherein the controller is configured to actuate the injection device when the actuation pressure has been exceeded.

12. The system of claim 9, wherein the pressure relief valve and the pressure sensor are an integral unit.

13. The system of claim 1, wherein the injection device and the sensing device are an integral unit.

14. The system of claim 1, and further comprising a check valve fluidly coupled to the catheter that is configured to prevent a negative pressure in the catheter.

15. The system of claim 14, wherein the check valve and the pressure relief valve are an integral unit.

16. The system of claim 1, wherein the sensing device is electrically, mechanically, or fluidly connected to the injection device.

17. The system of claim 1, wherein the pressure relief valve is a valve mechanism that comprises:
a disposable part comprising:
a body having a cavity, a first end, and a second end opposite the first end;
a tube extending through the first end of the body and into the cavity in the body, wherein a first end of the tube is configured to be fluidly coupled to the catheter; and
a first disk seal sealed in an opening at the second end of the body; and
a reusable part that is configured to releasably connect to the disposable part, the reusable part comprising:
a body having a cavity, a first end, and a second end opposite the first end;
a second disk seal sealed in an opening at the first end of the body;
a linear actuator in the cavity of the body; and
a spring extending between the linear actuator and the second disk seal.

18. The system of claim 17, wherein when the valve mechanism is in a closed position, the first disk seal is deformed and sealed against a second end of the tube in the disposable part, and wherein when the valve mechanism is in an open position, a second end of the tube is fluidly coupled to the cavity of the body of the disposable part.

19. The system of claim 1, wherein the injection device further comprises a syringe that is configured to inject fluid into the inflatable balloon of the catheter, wherein the syringe includes a barrel and a plunger positioned in the barrel, and a locking mechanism that is configured to lock the plunger of the syringe in position in the barrel of the syringe.

20. The system of claim 1, and further comprising:
the catheter having the inflatable balloon, wherein the inflatable balloon has a varying compliance from a proximal end to a distal end of the inflatable balloon.
